(19) United States
(12) Patent Application Publication
(10) Pub. No.: US 2003/0148454 A1
(43) Pub. Date: Aug. 7, 2003

(54) Virus-free vesicles for delivery of functional membrane bound proteins

(75) Inventors: Ann Marshak-Rothstein, Newton, MA (US); Shyr-Te Ju, Charlottesville, VA (US); Satoshi Jodo, Sapporo (JP); Andreas Michael Hohlbaum, Somerville, MA (US)

Correspondence Address: NIXON PEABODY LLP, 101 FEDERAL ST, BOSTON, MA, 02110, US

(73) Assignee: Trustees of Boston University

(21) Appl. No.: 10/287,278

(22) Filed: Nov. 4, 2002

Related U.S. Application Data
(60) Provisional application No. 60/336,279, filed on Nov. 2, 2001.

(51) Int. Cl. C12P 21/02; C12N 1/00; C12N 5/06; C07K 14/705; C07K 14/525

(52) U.S. Cl. 435/69.1; 530/351; 435/69.5; 435/320.1; 435/317.1; 435/325; 530/350

(57) ABSTRACT

The present invention is directed to methods for producing virus-free vesicles containing functional membrane proteins, and uses thereof. Preferably the virus-free vesicles contain a type II transmembrane protein which is a member of the TNF superfamily. The vesicles maintain the protein in its native conformation, thus eliminating the problems related to the solubility of these proteins when produced without presence of membrane component.

This Utility Application is based on Provisional Application 60/336,279, filed Nov. 2, 2001, the content of which is relied upon and incorporated herein by reference in its entirety, and benefit priority under 35 USC §119(e) is hereby claimed.

This invention was made with Government Support under Contract Nos. AI-36938, ES-10244, ES-06086, and GM-58724 awarded by the National Institutes of Health. The Government has certain rights in the invention.

### FIELD OF THE INVENTION

The present invention is directed to methods for preparing virus-free vesicles containing functional membrane-bound proteins and uses thereof. Preferably the virus-free vesicles contain a type II transmembrane protein which is a member of the TNF superfamily.

### BACKGROUND OF THE INVENTION

Programmed cell death is a physiologic process that ensures homeostasis is maintained between cell production and cell turnover in essentially all self-renewing tissues. In many cases, characteristic morphological changes, termed “apoptosis,” occur in a dying cell. Since similar changes occur in different types of dying cells, cell death appears to proceed through a common pathway in different cell types.

In addition to maintaining tissue homeostasis, apoptosis also occurs in response to a variety of external stimuli, including growth factor deprivation, alterations in calcium levels, free-radicals, cytotoxic lymphokines, infection by some viruses, radiation and most chemotherapeutic agents. Thus, apoptosis is an inducible event that likely is subject to similar mechanisms of regulation as occur, for example, in a metabolic pathway. In this regard, dysregulation of apoptosis also can occur and is observed, for example, in some types of cancer cells, which survive for a longer time than corresponding normal cells, and in neurodegenerative diseases where neurons die prematurely. In viral infections, induction of apoptosis can figure prominently in the pathophysiology of the disease process.

Several transmembrane molecules are involved in regulation of apoptosis. For example, CD95 or Fas-ligand, a type II transmembrane protein belonging to a TNF superfamily, induces apoptosis when it binds to Fas-expressing cells. TNF family of receptors include also at least two types of TNF receptors, Type I and II, (Smith et al., Science 248:1019, 1990; and Schall et al., Cell 61:361, 1990) nerve growth factor receptor (Johnson et al., Cell 47:545, 1986), B cell antigen CD40 (Stamenkovic et al., EMBO J. 8:1403, 1989), T cell antigen OX40 (Mallett et al., EMBO J. 9:1063, 1990), human Fas antigen (Itoh et al., Cell 66:233, 1991) and murine 4-1BB receptor (Kwon et al., Cell. Immunol. 121:414, 1989; and Kwon et al., Proc. Natl. Acad. Sci. USA 86:1963, 1989). In addition, a protein known as TNF-related apoptosis-inducing ligand (TRAIL) is a member of the tumor necrosis factor family of ligands (Wiley et al., Immunity, 3:673-682, 1995). TRAIL has demonstrated the ability to induce apoptosis of certain transformed cells, including a number of different types of cancer cells as well as virally infected cells (PCT application WO 97/01633 and Wiley et al., supra).

TNF superfamily members usually share a number of common features. These features do not include a high degree of overall amino acid (aa) sequence homology. With the exception of nerve growth factor (NGF) and TNF-beta, all ligands are synthesized as type II transmembrane proteins (extracellular C-terminus) that contain a short cytoplasmic segment (10-80 aa residues) and a relatively long extracellular region (140-215 aa residues). NGF, which is structurally unrelated to TNF, is included in this superfamily only because of its ability to bind to the TNFRSF low affinity NGF receptor (LNGFR). NGF has a classic signal sequence peptide and is secreted. TNF-beta, in contrast, although also fully secreted, has a primary structure much more related to type II transmembrane proteins. TNF-beta might be considered as a type II protein with a non-functional, or inefficient, transmembrane segment. In general, TNFSF members form trimeric structures, and their monomers are composed of beta-strands that orient themselves into a two sheet structure. As a consequence of the trimeric structure of these molecules, it is suggested that the ligands and receptors of the TNSF and TNFRSF superfamilies undergo “clustering” during signal transduction.

Due to these molecules' involvement in cellular processes associated with immune responses, tumorigenesis and other disease states, methods affecting their interactions would provide a novel therapeutics for immunosuppressive and/or anti-inflammatory and/or anticancer agents with activity towards (1) autoimmune disorders such as multiple sclerosis, systemic lupus erythematosus, or Graves' disease, and undesired immune responses, such as, for example, those that occur in graft versus host disease (GVHD); (2) a variety of inflammatory diseases or disorders with an inflammatory or T cell-mediated component such as various forms of arthritis; allograft rejections; asthma; inflammatory diseases of the bowel, including Crohn's disease; various dermatological conditions such as psoriasis; and the like, and (3) a variety of hyperproliferative diseases or disorders including, but not limited to, cancers, tumors, and the growth and spreading (metastasis) thereof.

The study of membrane proteins is more complicated than for water-soluble proteins, which can be readily purified in aqueous buffers and maintained in a native conformation. Membrane proteins in contrast cannot be solubilized in aqueous buffers but must be maintained in an environment that allows the membrane-spanning region of the protein to maintain its hydrophobic contacts. A critical problem with identifying molecules that can inhibit or enhance the function of these is transmembrane or membrane-bound proteins, and polypeptides is that the proteins are often insoluble in aqueous solution, limiting their use in screening assays. The same problem often limits the use of membrane bound proteins for therapeutic use. A method using retroviral particles to produce active Fas-ligand has been described (Jodo et al. Journal of Immunology 164:5062-5069). However, use of non-viral particles would be desirable, especially in therapeutic applications.

Accordingly, it would be desirable to have improved methods to produce and isolate membrane proteins, including type II transmembrane proteins which are members of the TNF superfamily, while maintaining the protein in its native conformation, thus eliminating the problems related to the solubility of these proteins when produced without presence of membrane component.

### SUMMARY OF THE INVENTION

The present invention is directed to methods for producing virus-free vesicles containing functional membrane proteins, and uses thereof. Preferably the virus-free vesicles contain a type II transmembrane protein which is a member of the TNF superfamily. The vesicles maintain the protein in its native conformation, thus eliminating the problems related to the solubility of these proteins when produced without presence of membrane component.

The method of the present invention for producing vesicles containing a functional membrane protein has the following steps: introducing a nucleic acid encoding the membrane protein into a cell capable of producing vesicles; selecting cells that express the nucleic acid; growing the cells to allow expression of the nucleic acid; collecting the culture medium; centrifuging the culture medium to remove cellular debris from the cell-free supernatant; centrifuging the cell-free supernatant to pellet the vesicles; and discarding the supernatant and resuspending the pellet containing the isolated vesicles containing the membrane protein in an isotonic solution.

Preferably, the membrane protein is a type II transmembrane protein which is a member of the tumor necrosis factor (TNF) superfamily, including Fas-ligand, TRAIL, RANKL, TWEAK, TNF-α, LTD, LIGHT, LTα1β2, TL1A, CD40-ligand, CD30-ligand, CD27L, 4-1BBL, OX40L, GITRL, APRIL, BAFF, EDA1, and EDA2. In one preferred embodiment, the protein is Fas-ligand. In another preferred embodiment, the protein is tumor necrosis factor or TRAIL.

The vesicles of the present invention are produced by using cell lines which allow production of virus-free vesicles to express the membrane protein of interest. One screens the transfected cell lines for production of vesicles which contain the protein of interest. Preferred cell lines for production of vesicles containing FasL include neuroblastoma cell lines such as N2, 3 T3 cells, and retroviral packaging cell lines such as PA317. In one preferred embodiment, N2 cells are used to produce vesicles containing FasL.

In another preferred embodiment, the vesicle comprises at least two functional membrane proteins.

### BRIEF DESCRIPTION OF THE FIGURES

FIG. 1 shows the effect of NOK-1 anti-FasL mAb on FasL VP-mediated cytotoxicity against LB27.4, LF+, M59, Jurkat, and K3 targets. Cytotoxicity was assessed in either a 5-hour or a 16-hour cytotoxicity assay, as dictated by target sensitivity to FasL VP. FasL VP was used at a concentration capable of inducing between 40-60% specific lysis of each target population. Various amounts of NOK-1 mAb were added at the beginning of the cytotoxicity assays. Mouse Ig control did not affect the cytotoxicity in all cases (not shown for clarity purpose). All assays were carried out in duplicate. All experiments were conducted between 3 to 10 times with similar results.

FIG. 2 shows that FasL VP prepared from N2-mFasL but not L5-mFasL express potent cytotoxicity that is modulated by Kay-10 anti-mouse FasL mAb. Cells, FasL VP, and supernatant (sup) following ultracentrifugation were prepared from N2-mFasL and L5-mFasL cells as described in Materials and Methods. Cytotoxicity of each preparation was assessed on LB27.4 target for cell-associated FasL (2a), supernatants (sup) (2b), and FasL VP (2c). In FIG. 2d, the ability of Kay-10 anti-FasL mAb to modulate the cytotoxicity of FasL VP prepared from N2-mFasL was assessed on LB27.4 and Jurkat targets.

FIG. 3 shows the effect of anti-FasL mAb on cytotoxicity of cell-associated FasL or sFasL on LB27.4 target. Various concentrations of NOK-1 mAb or Kay-10 mAb were added in the cytotoxicity assays in which soluble human FasL (sFasL), hFasL-3T3 cells or L5-mFasL cells were used against LB27.4 target. The cell number and the amount of sFasL were pre-determined such that approximately 40-60% specific lysis would be obtained after 5 hours in culture.

FIG. 4 shows that enhancement of FasL VP-mediated cytotoxicity depends on target FcR expression. In FIG. 4A, various doses of FasL VP were used to kill FcR−A20 and FcR+A20 targets in the presence or absence of NOK-1 mAb (10 ng/ml). In FIG. 4B, the ability of various doses of NOK-1 mAb to modulate FasL VP cytotoxicity was compared between the FcR+A20 and FcR- A20 targets.

FIG. 5 shows NOK-1-mediated enhancement but not inhibition of cytotoxicity was blocked by anti-FcR mAb. In FIG. 5A, various amounts of 2.4G2 or control rat IgG2b were added at the beginning of culture using 10 ng/ml NOK-1 to enhance the FasL VP cytotoxicity against LB27.4 target in a 5-hour cytotoxicity assay. FIG. 5B, a fixed amount (500 ng/ml) of 2.4G2 or rat IgG2b was used to determine its effect on enhancement and inhibition of cytotoxicity induced by various doses of NOK-1 mAb. In the absence of NOK-1, 2.4G2 had no effect on the FasL VP cytotoxicity against LB27.4.

FIG. 6 shows the effect of anti-FasL mAb subclass on the enhancement and inhibition of FasL VP-mediated cytotoxicity. Various doses of anti-FasL mAb were tested on their ability to modulate the cytotoxicity of FasL VP in a 5-hour cytotoxicity assay against LB27.4 target cells. The mAb tested were NOK-2 (IgG2a), Alf-1.2 (IgG), NOK-3 (IgM), and G247-4 (IgG1).

FIG. 7 shows that G247-4 anti-FasL mAb possesses the ability to inhibit FasL VP-mediated cytotoxicity. Various doses of G247-4 anti-FasL mAb were tested for their ability to modulate the cytotoxicity of FasL VP in a 5-hour assay against Jurkat target cells (FIG. 7A) or LB27.4 target cells (FIG. 7B). The ability of G247.4 mAb to inhibit FasL-mediated cytotoxicity against LB27.4 was determined by adding 2.4G2 mAb to block FcR-mediated enhancement. Cytotoxicity assay was conducted in the absence or presence of 0.5 μg/ml of 2.4G2 mAb. Rat IgG2b was used as a specificity control.

FIG. 8 shows the effect of Fas-IgG1 fusion protein on FasL VP-mediated cytotoxicity. Various doses of Fas-IgG1 were tested for their ability to modulate the cytotoxicity of FasL VP in a 5-hour assay against LB27.4 and Jurkat targets. Cytotoxicity assays were conducted in the absence and presence of 500 ng/ml of 2.4G2 mAb. Rat IgG2b was used as a specificity control.

FIG. 9 is a schematic depicting the method for vesicle purification according to the present invention.

FIG. 10 shows that FasL VP prepared from N2-mFasL cells, but not L5 mFasL cells, express potent cytotoxicity. Target B lymphoma cells (LB27.4) were labeled with radioactive Cr and then seeded into assay wells that contained either FasL transfected cells (left panel) or vesicles prepared from the culture supernatant of transfected cells (right panel). The extent of target cell death was assessed after a 4 hr incubation at 37° C. by quantifying Cr release into the culture medium. As shown in FIG. 12A, the cells from the two transfected lines exhibited comparable amounts of cytotoxic activity. By contrast, as shown in FIG. 12B1, vesicle preparations derived from the two lines had very different levels of cytotoxic activity.

FIG. 11 shows in vivo apoptosis of peritoneal macrophages in response to mFasL-VP. A/J mice were injected with 14 U of mFasL-VP or neo-VP and sacrificed after 30 min, 1, 2, or 4 h. Aliquots of isolated PEC and PWC (0 h) were triple-stained with Mac1, Gr1, and F4/80 or double-stained with annexin V and F4/80 and analyzed by flow cytometry. FIG. 14A, Mac1 vs F4/80 contour plot of Gr1-cells. FIG. 14B, Annexin V vs F4/80 contour plot.

FIG. 12 shows that in vivo inoculation of mFasL-VP elicits an inflammatory response. A/J mice were injected i.p. with 7 U of mFasL-VP or neo-VP and sacrificed after 4 or 18 h. In FIG. 12A, isolated PEC or PWC from the experimental mice and an unmanipulated A/J mouse (0 h) were stained with Mac1 and GR1 and analyzed by flow cytometry. In FIGS. 12B-C, RNA was isolated from the experimental and control cells and screened for the expression of murine proimflammatory cytokines and chemokines by RNase protection assays using the multiple template probe sets mCK2b and mCK5b (BD Phar-Mingen). One representative experiment from three independent experiments is shown. In FIG. 12B, results for MIP 1beta, MIT 1alpha, MIP2, and MCP-1 are shown, with the control L32. In FIG. 23C, results for IL 1beta and IL1-RA are shown, with the control L32.

FIG. 13 is a photo of a Western blot showing full-length FasL, but not other membrane proteins (such as a single chain anti-CD3), can be found in the microvesicles released by transfected Neuro2a cells. Western blot analysis of cell lysates (C) and vesicle preparations (V) of Neuro2a cells transfected with: FasL (lanes 1,2); vector control (lanes 3,4); single chain anti-CD3 (lanes 5,6); FasL and anti-CD3 (lanes 7,8). It is apparent that FasL can be detected in the cell lysates and the vesicle preparations of cells transfected with FasL. The single chain anti-CD3 can only be detected in the cell lysates of the cells transfected with anti-CD3. In data not shown, FasL and anti-CD3 can also be detected by flow cytometry on the cell membrane of the corresponding cell lines.

### DESCRIPTION OF THE INVENTION

The present invention is directed to methods for producing virus-free vesicles containing functional membrane proteins, and uses thereof. Preferably the vesicles contain a type II transmembrane protein which is a member of the TNF superfamily. The vesicles maintain the protein in its native conformation, thus eliminating the problems related to the solubility of these proteins when produced without presence of membrane component.

The method for producing virus-free vesicles comprises producing an expression cell line, collecting supernatant from the expression cell line, and isolating vesicles from the supernatant. The term “expression cell line” as used herein means any cell that is capable of excreting vesicles. In a preferred embodiment N2 and NIH-3T3 cells are used.

The expression cell line can be produced using genetic engineering techniques to introduce the nucleic acid which encodes the membrane protein of interest and allows its expression. In one preferred embodiment, the protein is Fas-ligand. In another preferred embodiment, the protein is tumor necrosis factor, CD40L, or TRAIL.

Cell lines which can be used to produce the expression cell line as used herein can be any cell line that is capable of excreting vesicles when a nucleic acid encoding a membrane protein is introduced into the cell line. In a preferred embodiment N2 and NIH-3T3 cells are used. To identify cell lines which allow production of vesicles containing the membrane protein of interest, one screens the transfected cell lines for production of vesicles which contain the protein of interest. Preferred cell lines for production of vesicles containing FasL include but are not limited to neuroblastoma cell lines such as N2, fibroblast cell lines such as NIH-3T3 cells, and retroviral packaging cell lines such as PA317. In one preferred embodiment, N2 cells are used to produce vesicles containing FasL.

The method of the present invention for isolating vesicles from the expression cell line's supernatant has the following steps: growing the cells to allow expression of the nucleic acid; collecting the culture medium; centrifuging the culture medium to remove cellular debris from the cell-free supernatant; centrifuging the cell-free supernatant to pellet the vesicles; and discarding the supernatant and resuspending the pellet containing the isolated vesicles containing the membrane protein in an isotonic solution.

Any method of expression may be used to express the desired membrane protein, prior to its isolation by the method of the present invention.

The expression cell line is cultured for an appropriate time to allow expression of the membrane protein, e.g., for about 1-5 days, preferably 2 days.

In certain embodiments, the expression cell line is adherent, and thus few if any cells are harvested from the culture medium is collected. In other embodiments, the expression cell is unattached and thus the cells are also harvested when the culture medium is collected. For both adherent and non-adherent cells, an initial cell-removing centrifugation can be performed prior to the centrifugation to remove cellular debris. Any centrifugation conditions which allow separation of these two fraction can be used. For example, the initial cell-removing centrifugation may be a 5 minute spin at 1100 rotations per minute (rpm), to roughly separate the intact cells from the culture medium containing the cellular debris and vesicles.

The first centrifugation step of the culture medium is to remove cellular debris (which is concentrated in the pellet fraction) from the cell-free supernatant, which contains the vesicles. Any centrifugation conditions which permit separation of these two fractions can be used, for example a 30 minute spin at 13,000 rpm.

The second centrifugation step of the cell-free supernatant is to separate the vesicles, which now concentrate in the pellet fraction, from the aqueous supernatant. Any conditions which permit separation of these two fractions ban be used, for example a 3-5 hour spin at 25,000 rpm.

To isolate the vesicles, the supernatant from the final centrifugation step is removed, leaving the pellet fraction containing the vesicles. The vesicles can be resuspended in any isotonic solution desired.

In a further embodiment of the present invention, the resuspended vesicle fraction may be further purified by filtration e.g. through a 0.45 micron filter, to produce a sterile preparation of vesicles.

Alternatively, the medium can be centrifuged using filters such as Centricon-50, Centricon-100 or Centricon-500 (Millipore, Bedford, Mass.) that filter through proteins less than the cut-off value and concentrates the fraction with desired molecular weight. Preferably, the diameter of the vesicles is less than 0.50 microns, most preferably about 0.45 microns. Preferably, this portion is then subjected to a functional assay for at least one function of the protein to ensure that the vesicles maintain the protein in their native conformation.

The method of the present invention can be used to produce any membrane protein of interest. Preferably, the membrane protein is a type II transmembrane protein which is a member of the tumor necrosis factor (TNF) superfamily. Members of the TNF superfamily include but are not limited to Fas-ligand, TRAIL, RANKL, TWEAK, TNF-α, LTα, LIGHT, LTα1β2, TL1A, CD40-ligand, CD30-ligand, CD27L, 4-1BBL, OX40L, GITRL, APRIL, BAFF, EDA1, and EDA2. In one preferred embodiment, the protein is Fas-ligand. In another preferred embodiment, the protein is tumor necrosis factor, CD40L, or TRAIL.

TNF-related ligands usually share a number of common features. These features do not include a high degree of overall amino acid (aa) sequence homology. With the exception of nerve growth factor (NGF) and TNF-beta, all ligands are synthesized as type II transmembrane proteins (extracellular C-terminus) that contain a short cytoplasmic segment (10-80 aa residues) and a relatively long extracellular region (140-215 aa residues). 7 NGF, which is structurally unrelated to TNF, is included in this superfamily only because of its ability to bind to the TNFRSF low affinity NGF receptor (LNGFR). NGF has a classic signal sequence peptide and is secreted. TNF-beta, in contrast, although also fully secreted, has a primary structure much more related to type II transmembrane proteins. TNF-beta might be considered as a type II protein with a non-functional, or inefficient, transmembrane segment. In general, TNFSF members form trimeric structures, and their monomers are composed of beta-strands that orient themselves into a two sheet structure. As a consequence of the trimeric structure of these molecules, it is suggested that the ligands and receptors of the TNSF and TNFRSF superfamilies undergo “clustering” during signal transduction.

Fas ligand (FasL) is a highly conserved, 40 kDa transmembrane glycoprotein that occurs as either a membrane bound protein or a circulating homotrimer. In humans, FasL is synthesized as a 281 aa residue protein with an 80 aa residue cytoplasmic region, a 22 aa residue transmembrane segment, and a 179 aa residue extracellular domain. When proteolytically cleaved, FasL is a 70 kDa homotrimer composed of 26 kDa monomers with full biological activity. In mice, the FasL is somewhat different. Although mouse FasL molecule has 77% aa sequence identity with human FasL, polymorphisms exist in the mouse FasL, leading to functionally distinct FasL forms. In addition, a one aa residue substitution at position 273 (Phe to Leu) results in the gld/gld (generalized lymphoproliferative disease) mutation. Finally, while FasL in a membrane-bound form shows species cross-reactivity, soluble mouse FasL is apparently biologically inactive. Cells known to express FasL include type II pneumocytes and bronchial epithelium, monocytes, LAK cells and NK cells, dendritic cells, B cells, macrophages, CD4+ and CD8+ T cells, and colon and lung carcinoma cells.

Human NGF is a 12.5 kDa, nonglycosylated polypeptide 120 aa residues long. Synthesized as a prepropeptide, there is an 18 aa residue signal sequence, a 103 aa residue N-terminal pro-sequence, and a 120 aa residue mature segment. Human to mouse, there is 90% aa sequence identity in the mature segment. In the mouse, NGF is referred to as beta-NGF, due to the existence of NGF in a 130 kDa (7S) heterotrimeric (abg) complex in submaxillary glands. Many cells, however, do not synthesize all the components of this 7S complex, and the typical form for NGF is a 25 kDa, non-disulfide linked homodimer. NGF and all other neurotrophins bind to the LNGFR, a member of the TNFRSF.

Human CD40L is a 39 kDa, type II (extracellular C-terminus) transmembrane glycoprotein that was originally identified on the surface of CD4+ T cells. With a predicted molecular weight of 29 kDa, CD40L is 261 aa residues long, with a 22 aa residue cytoplasmic domain, a 24 aa residue transmembrane segment, and a 215 aa residue extracellular region. Human to mouse, CD40L is 73% identical at the aa sequence level and mouse CD40L is apparently active in humans. Although usually considered to be a membrane bound protein, natural, proteolytically cleaved 15-18 kDa soluble forms of CD40L with full biological activity have also been described. Like TNF-alpha, CD40L is reported to form natural trimeric structures. Cells known to express CD40L include B cells, CD4+ and CD8+ T cells, mast cells and basophils, eosinophils, dendritic cells, and monocytes, NK cells, and gd T cells.

Mouse 4-1 BBL is a 50 kDa, 309 aa residue transmembrane glycoprotein that is the largest of the TNFSF members. With a predicted molecular weight of 34 kDa, the molecule has an 82 aa residue cytoplasmic region, a 21 aa residue transmembrane segment, and a 206 aa residue extracellular domain. Although human and mouse 4-1BB molecules exhibit 60% identity at the aa level, human and mouse 4-1BBL molecules exhibit only 36% identity at the aa level. This level of cross species conservation is much lower than that shown by other members of the TNFSF. In mice, two ligands are known for 4-1BB: 4-1BBL and laminin. Cells known to express 4-1BBL include B cells, dendritic cells, and macrophages.

Human TNF-alpha is a 233 aa residue, nonglycosylated polypeptide that exists as either a transmembrane or soluble protein. When expressed as a 26 kDa membrane bound protein, TNF-alpha consists of a 29 aa residue cytoplasmic domain, a 28 aa residue transmembrane segment, and a 176 aa residue extracellular region. The soluble protein is created by a proteolytic cleavage event via an 85 kDa TNF-alpha converting enzyme (TACE), which generates a 17 kDa, 157 aa residue molecule that normally circulates as a homotrimer. Normal levels of circulating TNF are reported to be in the 10-80 pg/mL range. While both membrane-bound and soluble TNF-alpha are biologically active, soluble TNF-alpha is reported to be more potent. Mouse to human, full-length TNF-alpha shows 79% aa sequence identity. Unlike human TNF-alpha, mouse TNF-alpha is glycosylated. The variety of cell types known to express TNF-alpha is enormous and includes macrophages, CD4+ and CD8+ T cells, adipocytes, keratinocytes, mammary and colon epithelium, osteoblasts, mast cells, dendritic cells, pancreatic beta-cells, astrocytes, neurons, monocytes, and steroid-producing cells of the adrenal zona reticularis.

OX40, the receptor for OX40L, is a T cell activation marker with limited expression that seems to promote the survival (and perhaps prolong the immune response) of CD4+ T cells at sites of inflammation. OX40L also shows limited expression. Currently only activated CD4+, CD8+ T cells, B cells, and vascular endothelial cells have been reported to express this factor. The human ligand is a 32 kDa, 183 aa residue glycosylated polypeptide that consists of a 21 aa residue cytoplasmic domain, a 23 aa residue transmembrane segment, and a 139 aa residue extracellular region. When compared to the extracellular region of TNF-alpha, OX40L has only 15% aa sequence identity, again emphasizing the importance of secondary and tertiary structures as the basis for inclusion in the TNF Superfamily. Human OX40L is 46% identical to mouse OX40L at the aa sequence level. Mouse OX40L is active in humans, but human OX40L is inactive in mice. Consistent with other TNFSF members, OX40L is reported to exist as a trimer.

Human CD27L is a 50 kDa, 193 aa residue type II (extracellular C-terminus) transmembrane glycoprotein that appears to have a very limited immune system expression pattern. Having less than 25% aa sequence identity to TNF-alpha and CD40L, the molecule has only a 20 aa residue cytoplasmic segment, an 18 aa residue transmembrane domain, and a 155 aa residue extracellular region. Although the 20 aa residue cytoplasmic segment is short by most standards, there is a suggestion that it has a signaling function, perhaps activating the cytolytic program of gd T cells and/or contributing necessary signals for antibody production in B cells. Cells known to express CD27L are usually activated cells and include NK cells, B cells, CD45RO+, CD4+ and CD8+ T cells, gd T cells, and certain types of leukemic B cells.

Human CD30L is a 40 kDa, 234 aa residue transmembrane glycoprotein with 72% aa sequence identity to its mouse counterpart. With a predicted molecular weight of 26 kDa, the molecule consists of a 46 aa residue cytoplasmic region, a 21 aa residue transmembrane segment, and a 172 aa residue extracellular domain. Species cross-reactivity has been reported. As suggested for CD27L, the cytoplasmic region is suggested to transduce a signal. The CD30/CD30L system is complex since CD30 ligation can induce both proliferation and apoptosis. Cells known to express CD30L include monocytes and macrophages, B cells plus activated CD4+ and CD8+ T cells, neutrophils, megakaryocytes, resting CD2+ T cells, erythroid precursors, and eosinophils.

TNF-beta, otherwise known as lymphotoxin-alpha (LT-alpha) is a molecule whose cloning was contemporary with that of TNF-alpha. Although TNF-beta circulates as a 171 aa residue, 25 kDa glycosylated polypeptide, a larger form has been found that is 194 aa residues long. The human TNF-beta cDNA codes for an open reading frame of 205 aa residues (202 in the mouse), and presumably some type of proteolytic processing occurs during secretion. As with TNF-alpha, circulating TNF-beta exists as a non-covalently linked trimer and is known to bind to the same receptors as TNF-alpha. Circulating TNF-beta levels are reported to be about 150 pg/mL. Human TNF-beta is 72% identical to mouse TNF-beta at the aa sequence level across the entire molecule. TNF-alpha to TNF-beta, aa sequence identity is reported to be 28%. Unlike TNF-alpha, TNF-beta does not have a transmembrane form. However, it can be membrane-associated, due to its binding to membrane-anchored LT-beta (see below). In this complex, TNF-beta and LT-beta will form a heterotrimer that binds to both the LT-beta receptor and TNFRI receptor. Activation of the TNFRI receptor, however, does not occur. Cells known to express TNF-beta include NK cells, T cells and B cells.

Human lymphotoxin-beta (LT-beta), also known as p33, is a 33 kDa type II (extracellular C-terminus) transmembrane glycoprotein originally cloned from a T cell hybridoma cell line. It is 244 aa residues long, and has a 16 aa residue cytoplasmic segment, a 31 aa residue transmembrane domain, and a 197 aa residue extracellular region. On the membrane surface, LT-beta readily forms a trimeric complex with TNF-beta, in either a 2:1 (major form) or a 1:2 (minor form) ratio. LT-beta is not secreted. A comparison of human to mouse LT-beta shows 80% aa sequence identity in homologous regions. Overall, however, the mouse gene shows significant differences from the human gene. In mice, an intron has been incorporated into the genome creating a 66 aa residue insert into what would otherwise be a 240 aa residue molecule.

TRAIL, or TNF-related apoptosis-inducing ligand, is a newly discovered TNFSF member initially cloned from human heart and lymphocyte cDNA libraries. With a predicted molecular weight of 32 kDa, human TRAIL is 281 aa residues long, with a 17 aa residue cytoplasmic tail, a 21 aa residue transmembrane segment, and 243 aa residue extracellular region. Human TRAIL is 65% identical to mouse TRAIL at the aa sequence level across the entire molecule and there is complete species cross-reactivity. As a membrane bound protein, TRAIL shows a trimeric structure. Although TRAIL is known to be expressed by lymphocytes, many tissues seem to express the ligand, and this broad expression pattern suggests an intriguing function for the molecule.

Sequences of these proteins are widely available in the literature and from computer databases such as Genbank. Thus, one can readily obtain the gene encoding a particular protein of interest. This gene can be expressed by any known means. These include creating an expression cassette, where the gene is operably linked to a promoter. Other enhancing elements are known and may also be used. The codons used to synthesize the protein of interest may be optimized, converting them to codons that are preferentially used in mammalian cells. Optimal codons for expression of proteins in non-mammalian cells are also known, and can be used when the host cell is a non-mammalian cell (for example, insect cells, yeast cells, bacteria).

The nucleic acid encoding a protein of interest may contain any further useful modifications. For example, in one embodiment, any protein may be “tagged” to attach to the vesicle. For example, posttranslational glycosylphosphatidylinositol (GPI) anchor attachment serves as a general mechanism for linking proteins to the cell surface membrane (Online Mendelian Inheritance In Man database *603048). The GPI anchor attachment site is encoded by a conserved DNA sequence and can be cloned from proteins such as squid Sgp1 and 2 (U.S. Pat. No. 6,130,061).

For example, a nucleic acid construct encoding a fusion-protein of GPI and the protein desired to be included on the vesicles of the present invention can be introduced into the expression cell line thereby creating a cell line expressing vesicles with two different desired membrane-bound molecules.

The nucleic acid encoding the desired membrane protein can be introduced into the expression cell line by any conventional method. In one embodiment, two or more different nucleic acids are introduced into the expression cell line thus producing a bifunctional vesicle. For example, in the bifunctional vesicles of the present invention, one membrane-bound protein may be directed to target the target cell and another membrane-bound protein may be directed to induce apoptosis.

The gene or gene is introduced into a cell for the expression by methods known in the art. These methods include, e.g., vectors, liposomes, naked DNA, adjuvantassisted DNA, gene gun and catheters. Vectors include, e.g., chemical conjugates, plasmids and phage. The vectors can be chromosomal, non-chromosomal or synthetic. Commercial expression vectors are well known in the art, for example, vectors such as pcDNA 3.1, pcDNA4 HisMax, pACH, pMT4, PND are routinely used. Promoters that can be used to express the gene are also well known in the art. The promoter chosen are selected based upon the host cell which the protein is expressed in. These include, e.g., cytomegalovirus (CMV) intermediate early promoter, a viral LTR such as the Rous sarcoma virus LTR, HIV-LTR, HTLV-1 LTR, the simian virus 40 (SV40) early promoter, E.coli lac UV5 promoter and the herpes simplex tk virus promoter.

Preferred vectors include viral vectors, fusion proteins and chemical conjugates. Retroviral vectors include Moloney murine leukemia viruses. Other vectors include pox vectors such as orthopox or avipox vectors, herpesvirus vectors such as a herpes simplex I virus (HSV) vector (Geller, A. I. et al., (1995), J. Neurochem, 64: 487; Lim, F., et al., (1995) in DNA Cloning: Mammalian Systems, D. Glover, Ed., Oxford Univ. Press, Oxford England; Geller, A. I. et al. (1993), Proc Natl. Acad. Sci.: U.S.A. 90:7603; Geller, A. I., et al., (1990) Proc Natl. Acad. Sci USA 87:1149), adenovirus vectors (LeGal LaSalle et al. (1993), Science, 259:988; Davidson, et al. (1993) Nat. Genet 3: 219; Yang, et al., (1995) J. Virol. 69: 2004) and adeno-associated virus vectors (Kaplitt, M. G., et al. (1994) Nat. Genet. 8: 148). The particular vector chosen will depend upon the host cell used.

The introduction of the gene into the host cell can be by standard techniques, e.g. infection, transfection, transduction or transformation. Examples of modes of gene transfer include, e.g., naked DNA, CaPO4 precipitation, DEAE dextran, electroporation, protoplast fusion, lipofection, cell microinjection, and viral vectors.

An antigenic tag may be inserted in the protein to assist in its purification and in orienting the protein on the solid surface. Preferably, the tag is present at either the N-terminal end or the C-terminal end of the protein. The tag is preferably 6 to 15 amino acids in length, still more preferably about 6 to 9 amino acids. The tag is selected and its coding sequence inserted into the gene encoding the protein in a manner not to affect the overall conformation or function of the protein. Tags can include HA, polyoma, C9, FLAG, etc.

In one embodiment, the virus-free vesicles of the present invention can be used in screening assays to identify organic or inorganic compounds, peptides, peptidomimetics or proteins that interact with a membrane-bound protein. Preferably the screening methods are designed to identify compounds or molecules that are useful to regulate apoptosis.

In yet another embodiment, the virus-free vesicles carrying membrane-bound proteins of the present invention can be used as therapeutic agents for a variety of disorders as the virus-free vesicles can be engineered to contain tissue or cell specific targeting molecules. Preferably, the virus-free vesicles of the present invention are used to treat disorders that are related to abnormal apoptosis in cancer. For example, cancer cells typically express Fas. When Fas interacts with a virus-free vesicle carrying Fas-ligand, the cell undergoes apoptosis.

More preferably, the vesicles of the present invention are used to treat autoimmune diseases including but not limited to Alopecia Areata; Ankylosing Spondylitis; Antiphospholipid Syndrome; Autoimmune Addison's Disease; Autoimmune Hemolytic Anemia; Autoimmune Hepatitis; Behcet's Disease; Bullous Pemphigoid; Cardiomyopathy; Celiac Sprue-Dermatitis; Chronic Fatigue Immune Dysfunction Syndrome (CFIDS); Chronic Inflammatory Demyelinating Polyneuropathy; Churg-Strauss Syndrome; Cicatricial Pemphigoid; CREST Syndrome; Cold Agglutinin Disease; Crohn's Disease; Discoid Lupus; Essential Mixed Cryoglobulinemia; Fibromyalgia-Fibromyositis; Graves' Disease; Guillain-Barre Hashimoto's Thyroiditis; Idiopathic Pulmonary Fibrosis; Idiopathic Thrombocytopenia Purpura (ITP); IgA Nephropathy; Insulindependent Diabetes; Juvenile Arthritis; Lichen Planus; Lupus; Meniere's Disease; Mixed Connective Tissue Disease; Multiple Sclerosis; Myasthenia Gravis; Pemphigus Vulgaris; Pernicious Anemia; Polyarteritis Nodosa; Polychondritis; Polyglandular Syndromes; Polymyalgia Rheumatica; Polymyositis and Dermatomyositis; Primary Agammaglobulinemia; Primary Biliary Cirrhosis; Psoriasis; Raynaud's Phenomenon; Reiter's Syndrome; Rheumatic Fever; Rheumatoid Arthritis; Sarcoidosis Scleroderma; Sjogren's Syndrome; Stiff-Man Syndrome; Takayasu Arteritis; Temporal Arteritis/Giant Cell Arteritis; Ulcerative Colitis Uveitis; Vasculitis; Vitiligo; Wegener's Granulomatosis.

Most preferably the vesicles of the present invention are used to treat arthritis, systemic lupus erythematosus.

In another embodiment, the virus-free vesicles of the present invention may include two or more membrane-bound proteins on their surface.

The virus-free vesicles can be administered in pharmaceutically acceptable carrier using any method of administration including systemic oral, intravenous, intra-arterial, intramuscular, subcutaneous, peritoneal, intranasal, transdermal and inhalation. The invention also contemplates a pharmaceutical composition comprising virus-free vesicles.

In another embodiment the virus-free vesicles of the present invention carrying membrane bound proteins can be used as immunogens to produce antibodies against the membrane-bound proteins in the native form.

The term “antibodies” is meant to include monoclonal antibodies, polyclonal antibodies and antibodies prepared by recombinant nucleic acid techniques that are selectively reactive with polypeptides encoded by nucleotide sequences of the present invention. The term “selectively reactive” refers to those antibodies that react with one or more antigenic determinants on e.g. FasL and do not react with other polypeptides. Antigenic determinants usually consist of chemically active surface groupings of molecules such as amino acids or sugar side chains and have specific three dimensional structural characteristics as well as specific charge characteristics. Antibodies can be used for diagnostic applications or for research purposes, as well as to block binding interactions.

For preparation of antibodies directed toward the immunogenic vesicles, any technique that provides for the production of antibody molecules may be used.

For example, mice can be immunized twice intraperitoneally with approximately 50 micrograms of vesicle immunogen per mouse. Sera from such immunized mice can be tested for antibody activity by immunohistology or immunocytology on any host system expressing such polypeptide or against another vesicle or by ELISA with the expressed polypeptide. For immunohistology, active antibodies of the present invention can be identified using a biotin-conjugated anti-mouse immunoglobulin followed by avidin-peroxidase and a chromogenic peroxidase substrate. Preparations of such reagents are commercially available; for example, from Zymed Corp., San Francisco, Calif. Mice whose sera contain detectable active antibodies according to the invention can be sacrificed three days later and their spleens removed for fusion and hybridoma production. Positive supernatants of such hybridomas can be identified using the assays described above and by, for example, Western blot analysis.

Another method for preparing antibodies is by using hybridoma mRNA or splenic mRNA as a template for PCR amplification of such genes [Huse, et al., Science 246:1276 (1989)]. For example, intrabodies can be derived from murine monoclonal hybridomas [Richardson, J. H., et al., Biochem and Biophys Res Comm. 197: 422-427 (1993); Mhashilkar, A. M., et al., EMBO J. 14:1542-1551 (1995)]. These hybridomas provide a reliable source of well-characterized reagents for the construction of antibodies and are particularly useful when their epitope reactivity and affinity has been previously characterized. Another source for such construction includes the use of human monoclonal antibody producing cell lines [Marasco, W. A., et al., Proc. Natl. Acad. Sci. USA 90:7889-7893 (1993); Chen, S. Y., et al., Proc. Natl. Acad. Sci. USA 91:5932-5936 (1994)]. Another example includes the use of antibody phage display technology to construct new antibodies against different epitopes on a target molecule [Burton, D. R., et al., Proc. Natl. Acad. Sci. USA 88:10134-1-137 (1991); Hoogenboom, H. R., et al., Immunol. Rev. 130:41-68 (1992); Winter, G., et al., Ann. Rec. Immunol. 12:433-355 (1994); Marks, J. D., et al., J. Biol. Chem. 267:16007-16010 (1992); Nissim, A., et al., EMBO J. 13:692-698 (1994); Vaughan, T. J., et al., Nature Bio. 14:309-314 (1996); Marks, C., et al., New Eng. J. Med. 335: 730-733 (1996)]. For example, very large naive human sFV libraries have been and can be created to offer a large source of rearranged antibody genes against a plethora of target molecules. Smaller libraries can be constructed from individuals with autoimmune disorders [Portolano, S,. et al., J. Immunol. 151:2839-2851 (1993); Barbas, S. M., et al., Proc. Natl. Acad. Sci. USA 92:2529-2533 (1995)] or infectious diseases [Barbas, C. F., et al., Proc. Natl. Acad. Sci. USA 89:9339-9343 (1992); Zebedee, S. L., et al., Proc. Natl. Acad. Sci. USA 89:3175-3179 (1992)] in order to isolate disease specific antibodies.

Other sources include transgenic mice that contain a human immunoglobulin locus instead of the corresponding mouse locus as well as stable hybridomas that secrete human antigen-specific antibodies [Lonberg, N., et al., Nature 368:856-859 (1994); Green, L. L., et al., Nat. Genet. 7:13-21 (1994)]. Such transgenic animals provide another source of human antibody genes through either conventional hybridoma technology or in combination with phage display technology. In vitro procedures to manipulate the affinity and find specificity of the antigen binding site have been reported including repertoire cloning [Clackson, T., et al., Nature 352: 624-628); marks, J. D., et al., J. Mol. Biol. 222: 581-597 (1991); Griffiths, A.D., et al., EMBO J. 12: 725-734 (1993)], in vitro affinity maturation [Marks, J. D., et al., Biotech 10: 779-783 (1992); Gram, H., et al., Proc. Natl. Acad. Sci. USA 89: 3576-3580 (1992)], semisynthetic libraries [Hoogenboom, H. R., supra; Barbas, C. F., supra; Akamatsu, Y., et al., J. Immunol. 151: 4631-4659 (1993)] and guided selection [Jespers, L. S. et al., Bio Tech 12: 899902 (1994)]. Starting materials for these recombinant DNA based strategies include RNA from mouse spleens [Clackson, t., supra] and human peripheral blood lymphocytes [Portolano, S., et al., supra; Barbas, C. F., et al., supra; Marks, J. D., et al., supra; Barbas, C. F., et al., Proc. Natl. Acad. Sci. USA 88: 7978-7982 (1991)] and lymphoid organs and bone marrow from HIV-1 infected donors [Burton, D. R., et al., supra; Barbas, C. F., et al., Proc. Natl. Acad. Sci. USA 89:9339-9343 (1992)].

For preparation of monoclonal antibodies directed toward the vesicles, any technique that provides for the production of antibody molecules by continuous cell lines may be used. For example, the hybridoma technique originally developed by Kohler and Milstein (Nature, 256: 495-7,1973), as well as the trioma technique, the human B-cell hybridoma technique (Kozbor et al., Immunology Today 4:72), and the EBV-hybridoma technique to produce human monoclonal antibodies, and the like, are within the scope of the present invention. See, generally Larrick et al., U.S. Pat. No. 5,001,065 and references cited therein. Further, single-chain antibody (SCA) methods are also available to produce antibodies against polypeptides encoded by a eukaryotic nucleotide sequence of the invention (Ladner et al., U.S. Pat. Nos. 4,704,694 and 4,976,778).

The monoclonal antibodies may be human monoclonal antibodies or chimeric human-mouse (or other species) monoclonal antibodies. The present invention provides for antibody molecules as well as fragments of such antibody molecules.

Those of ordinary skill in the art will recognize that a large variety of possible moieties can be coupled to the resultant antibodies or preferably to the stabilized trimers or to other molecules of the invention. See, for example, “Conjugate Vaccines”, Contributions to Microbiology and Immunology, J. M. Cruse and R. E. Lewis, Jr (eds.), Carger Press, New York, 1989, the entire contents of which are incorporated herein by reference.

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, disocyanates, glutaraldehydes, diazobenzenes and hexamethylene diamines. This listing is not intended to be exhaustive of the various classes of coupling agents known in the art but, rather, is exemplary of the more common coupling agents (see Killen and Lindstrom, J. Immunol. 133:1335-2549, 1984; Jansen, F. K., et al., Imm. Rev. 62:185-216, 1982; and Vitetta et al., supra).

Preferred linkers are described in the literature. See, for example, Ramakrishnan, S., et al., Cancer Res. 44: 201-208 (1984), describing the use of MBS (M-maleimidobenzoyl-N-hydroxysuccinimide ester). See also Umemoto et al., U.S. Pat. No. 5,030,719, describing the use of a halogenated acetyl hydrazide derivative coupled to an antibody by way of an oligopeptide linker. Particularly preferred linkers include: (i) EDC (1-ethyl-3-(3-dimethylamino-propyl carboduimide hydrochloride; (ii) SMPT (4-succinimidyloxycarbonyl-alpha-methyl-alpha-(2-pyridyl-dithio)-toluene (Pierce Chem. Co., Cat. (21558G); (iii) SPDP (succinimidyl-6[3-(2-pyridyldithio) propionamido] hexanoate (Pierce Chem. Co., Cat. #21651G); (iv) Sulfo-LC-SPDP (sulfosuccinimidyl 6 [3-(2-pyridyldithio-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

Complexes that form with molecules of the present invention can be detected by appropriate assays, such as the direct binding assay discussed earlier and by other conventional types of immunoassays.

In a preferred embodiment, one could screen a phage display library looking to find antibodies to a given protein or find ligands that will bind to the protein.

One can also use these vesicles to screen libraries for a desired compound. One can also use these vesicles to screen complex chemical libraries of small molecular weight (<1000 daltons) compounds to identify high-affinity ligands. These compounds could serve as lead compounds for the discovery of agonistic and antagonistic drugs.

If one knows a ligand that interacts with the protein, one can use these vesicles in assays to screen for compounds that modulate such interactions with the protein. For example, in the aforementioned FasL-containing vesicles, one can add Fas to the mixture and add other compounds to see their effect on the formation or stability of the Fas/FasL complex.

One can also use this method to identify small antagonists in an assay that looks at compounds that affect binding of a known ligand

In one preferred embodiment, the vesicles of the present invention express Fas-ligand, as known as FasL. In vivo, expression of FasL can be inducible, for example in T cells where it plays a role in T cell cytotoxicity and activation induced cells death, and in nonlymphoid tissues such as systemic SEB and upon UV exposure. FasL is also expressed constitutively, for example at sites of immune privilege including the eye, testis, and placenta, as well as in certain tumors including melanoma, colon carcinoma, lung carcinoma, astrocytoma, and esophageal carcinoma, indicating a role for FasL in immune evasion. Other evidence indicates that FasL expression may protect allografts, including of myoblasts co-transplanted with islet cells, colon carcinoma, macrophages, and dendritic cells. Other evidence also indicates that FasL expression accelerates rejection, including of islet beta cells, colon carcinoma, lymphoma, ficrosarcoma, and neuroblastoma.

Therapeutic application of FasL, including FasL vesicles, sometimes referred to herein as FasL VP, include direct cytotoxicity of Fas+target cells and indirect elicitation of an inflammatory response. Examples of FasL induced expression and/or secretion of cytokines include during death and inflammation, such as vascular smooth muscle cells/chemokines, thioglycolate elicited neutrophils/IL-1b, and macrophages from Propionibactrium acnes primed mice/IL-18. FasL induced expression of cytokine secretion in the absence of cell death includes dendritic cells' secretion of IL1b, TNFa, colon carcinoma cells' secretion of IL-8, rheumatoid arthritis synoviocytes' secretion of IL-8, early passage astrocytes' secretion of IL-8, murine astrocytes' secretion of MIP-1b, MIP-1a, and MIP-2, and human peripheral blood monocytes' secretion of IL-10. FasL induced expression of cytokines also occurs during proliferation of T cells, fibroblasts, some tumor cells. and during liver regeneration after partial hepatectomy.

FasL vesicles are useful as an experimental tool for in vitro studies. For example, they are a potent physiologically relevant source of FasL to determine sensitivity of Fas+ target cells to FasL engagement, and to eliminate Fax+ cells from a mixed population either independent of adhesion molecules in T cells, and independent of FcR expression in antibodies. FasL vesicles are also a valuable source of FasL for analysis of Fas signaling pathways, with no reverse signaling artifacts (e.g. in T cells). FasL vesicles also have consistent activity from assay to assay, and frozen aliquots can be stored indefinitely. FasL vesicles are also potent agents for the removal of Fas+ target cells in vivo. FasL vesicles are also useful for targeting a proinflammatory agent in vivo, including co-incorporation of single chain antibodies to provide specific targeting capability, for example to specific tumor of T cell subpopulations. The vesicle technology of the present invention can also be used with other FasL family members in similar applications.

### EXAMPLE

### Materials and Methods

Production of FasL-expressing Cell Lines of hFasL-PA317, N2-mFasL, and L5-mFasL

A retroviral packaging cell line carrying the hFasL gene (hFasL-PA317) was prepared according to the method described by A. D. Miller (11). The hFasL cDNA (provided by Dr. S. Nagata, Osaka University Medical School, Japan) was cloned into pLXSN (A. D. Miller, Fred Hutchinson Cancer Research Center, Seattle, Wash.; accession #M28248) and the construct was transfected into the PE501 cells (obtained from A. D. Miller) with lipofectamine. The virus-laden supernatant was used to infect PA317 cells (ATCC, Manassas, Va.) for 24 hours followed by selection with G418 (0.4 mg/ml)-containing culture medium. Six cloned G418-resistant cell lines were derived. One clone was expanded and used in this study. A similarly prepared packaging cell line carrying the human cKrox gene (Krox-PA317) was used as control throughout the study (12).

The N2-mFasL cell line was prepared by electroporating the Neuro-2a tumor cell line (ATCC) with a mouse FasL gene whose metalloproteinase sensitive site had been deleted (13). We tested twenty G418 (0.8 mg/ml)-resistant clones and found nineteen of them to express strong cell-mediated cytotoxicity. A clone was expanded for use in this study. The L5-mFasL cell line, derived by transfecting L5178Y (ATCC) cells with the same mouse FasL gene, has also been described (13).

FasL VP

Cloned hFasL-PA317 and N2-mFasL cells were maintained in a 150 mm ×25 mm culture dish in 30 ml of G418-containing culture medium. When the cells reached approximately 70% confluence, fresh culture medium without G418 was used to replace the G418-containing medium. Supernatants were collected 48 hours later and centrifuged at 13,000 rpm in a Sorvall Superspeed centrifuge at 5° C. for 30 minutes to remove cell debris. To generate human FasL VP, the cell-free supernatants were centrifuged for 3 hours at 5° C. at 25,000 rpm in a Beckman ultracentrifuge using an SW25 rotor. The pellet was suspended with culture medium to 7% of the original volume and passed through a 0.45 pt sterile filter. Less than 10% of cytotoxic activity was lost after the filtration, which also defines the upper size limit of the bioactive vesicles. To generate human sFasL, the cell free supernatants were centrifuged for 18 hours at 5° C. at 25,000 rpm in a Beckman ultracentrifuge using an SW25 rotor, and the top 80% of the supernatant was collected. The biologic activities and the physiochemical properties of FasL VP and sFasL have been characterized in previous studies (9, 10). Both human FasL VP and human sFasL express cytotoxicity against LB27.4 target cells. The FasL VP have a density of 1.14-1.16 g/ml, contain full-length FasL, and are retained by a filter (Centriprep 500, Millipore, Burlington, Mass.) that excludes particles with molecular weights smaller than 500 kDa. In contrast, sFasL readily passes through Centriprep 500, but is retained by a filter that excludes particles with molecular weights less than 50 kDa (Centricon-50, Millipore). A previous study has shown that some sFasL exist as aggregates of 300 kDa (14).

The same procedures were used to prepare FasL VP from N2-mFasL cells that over-express mouse FasL lacking the metalloproteinase sensitive site. Following ultracentrifugation, the murine FasL VP displayed strong FasL-specific cytotoxicity and full-length FasL could be detected by Western blot using polyclonal rabbit anti-mouse FasL antibodies. Cytotoxic activity was not detected in the supernatant obtained following ultracentrifugation. In addition, sFasL was not detected in the supernatant by Western blot analysis. As described in a previous study (13), Western blot analysis detected sFasL in supernatants that were prepared from a cell line over-expressing the wildtype FasL (data not shown).

### Cytotoxicity Assays

Target cells, LB27.4 (B lymphoma hybridoma, ATCC), LF+ (T lymphoma, 15), K31H28 T cell hybridoma (K3, 16), M59 (macrophage hybridoma, 17), FcR-A20, FcR+ A20 (kindly provided by Dr. C. Janeway, Yale University, New Haven Conn., 18) or Jurkat (T lymphoma, obtained form Dr. R. Tepper, Mass. General Hospital) were labeled with Na251CrO4 as previously described (19). Various amounts of the FasL VP were cultured with 2×104 target cells, in a total of 0.2 ml in each well of a 96-well plate. At 5 hours or 16 hours after culture, supernatants were removed and counted with a □-scintillation counter. Use of the 5-hour and 16-hour cytotoxicity assays was based on the sensitivity of the various target populations under the specific assay conditions. Target cells cultured in the absence of FasL VP were used as background release. Target cells lysed with 0.5% NP40 were used as total release, which represents 100% cell death. Cytotoxicity is expressed as % specific 51Cr-release according to the formula: 100×(experimental release-background release)/(total release-background release). The effect of anti-FasL mAb was studied by adding various dilutions of the mAb at the beginning of culture. The anti-human FasL mAb tested were NOK-1 (mouse IgG1, PharMingen), NOK-2 (mouse IgG2a, PharMingen), NOK-3 (mouse IgM, provided by K. Okumura) (5), G247.4 anti-FasL mAb (mouse IgG1, PharMingen), and Alf-1.2 (mouse IgG, provided by D. Kaplan, Case Western Reserve University, Ohoi) (20). The isotype of Alf-1.2 has not been established. The anti-mouse FasL mAb Kay-10 was also used in these studies (mouse IgG2b, PharMingen, 21). Normal mouse isotypes were used as controls. The preparation of Fas-IgG1, a mouse Fas and human IgG1 fusion protein has been described (16). To determine the role of mouse FcR, 2.4G2 anti-FcR mAb (Rat IgG2b, PharMingen, 22) or normal rat IgG2b (PharMingen) control was added together with the anti-FasL mAb at the beginning of culture. All experiments were carried out in duplicate and conducted two times or more.

FcR Expression

Cell surface FcR expression on target cells was determined by Flow cytometry using biotin-conjugated 2.4G2 mAb. Cell bound 2.4G2 mAb was measured with PE-streptavidin. Biotin-conjugated rat IgG2b was used as a specificity control.

Results

The CD95 (Fas) ligand, FasL, is a type II transmembrane protein and a member of the TNF superfamily (1,2). FasL is expressed on various types of cells including activated T cells (1,2). When it interacts with Fas-expressing cells, cell-associated FasL cross-links Fas and the subsequent aggregation of Fas activates an apoptotic program leading to the death of the Fas-expressing cells (3,4). In addition to cell-associated FasL, a soluble form of FasL, sFasL, is produced as a result of metalloproteinase digestion (5-7). The sFasL has a limited target range. Under certain experimental conditions, sFasL can be inhibitory to cell-associated FasL (6,7).

FasL-expressing cells also produce microvesicles (FasL VP) that bear FasL and display FasL function (7, 8-10). We have recently shown that the FasL VP prepared from hFasL-PA317 cells (a retroviral packaging cell line for the human FasL gene) are highly cytotoxic and capable of killing sensitive targets within a 5-hour time period (9,10). In contrast to cell-associated FasL and sFasL, the FasL VP-mediated killing of the B lymphoma target LB27.4 can be enhanced by low doses of anti-FasL mAb (NOK-1, mouse IgG1), whereas high doses of NOK-1 inhibit the activity of all three forms of FasL (10). Cross-linking of FasL VP by a low dose of NOK-1 could work by generating complexes that consist of multiple bioactive vesicles. Such complexes could interact more effectively with the target cells. Alternatively, FcR expressed by the LB27.4 cells could bind the NOK-1/FasL VP complexes and increase the extent of interaction between FasL VP and target cells. The latter explanation implies that the enhancement of FasL VP-mediated cytotoxicity would depend on the subclass of anti-FasL mAb and the FcR expression level of the targets. Here, we examined six anti-FasL mAb, seven target cell lines, and FasL VP prepared from cell lines expressing either human FasL or murine FasL. The data presented herein establish that an FcR-dependent mechanism is responsible for the anti-FasL-mediated enhancement of FasL VP cytotoxicity. The anti-FasL mAb-mediated, FcR-dependent enhancement of FasL VP cytotoxicity represents a novel form of cytotoxicity previously unknown to the immune system. The significance, implication and utilization of the novel cytotoxic mechanism are discussed. [Ron I think we should delete this whole paragraph]

Enhancement but not inhibition of FasL VP-mediated cytotoxicity is target cell-dependent.

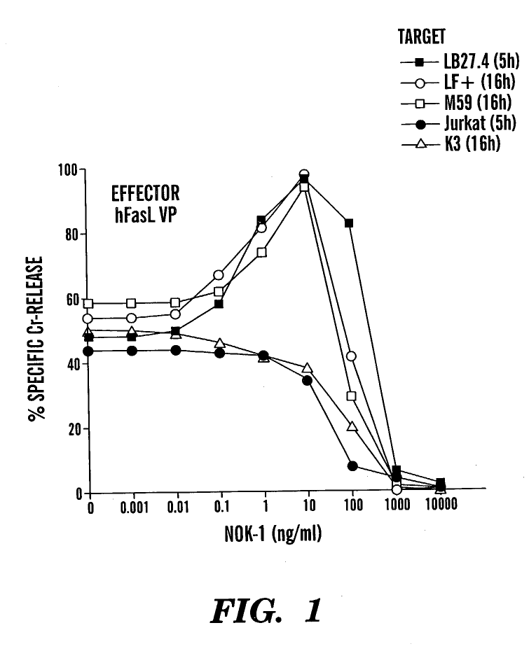

We determined the ability of NOK-1 anti-human FasL mAb to modulate the cytotoxicity mediated by cell-associated human FasL, FasL VP, and sFasL, prepared from the hFasL-PA317 cell line. Similar preparations from the control Krox-PA317 cell line were used to establish the specificity of these reagents (9, 10). Five targets (LB27.4, LF+, M59, Jurkat, and K3) frequently used in our laboratory were compared. We first tested the effect of NOK-1 on FasL VP cytotoxicity (FIG. 1). For each target we used a concentration of FasL VP that induced between 40-60% specific lysis of that target for the analysis. In the presence of high concentrations of NOK-1 (1-10 μg/ml), target death induced by FasL VP was strongly inhibited. As the NOK-1 concentration decreased, a dose-dependent reduction of inhibition was observed. Interestingly, as the concentration decreased further, a significant enhancement of killing was observed with LB27.4, LF+ and M59, but not with Jurkat and K3 target cells. The latter observation indicates that cross-linking of FasL VP by NOK-1 is not sufficient to enhance cytotoxicity. The “bell-shaped” enhancement response was observed in a dose range between 1100 ng/ml. Under identical condition, no effect on cytotoxicity was observed with isotype control antibodies (not shown).

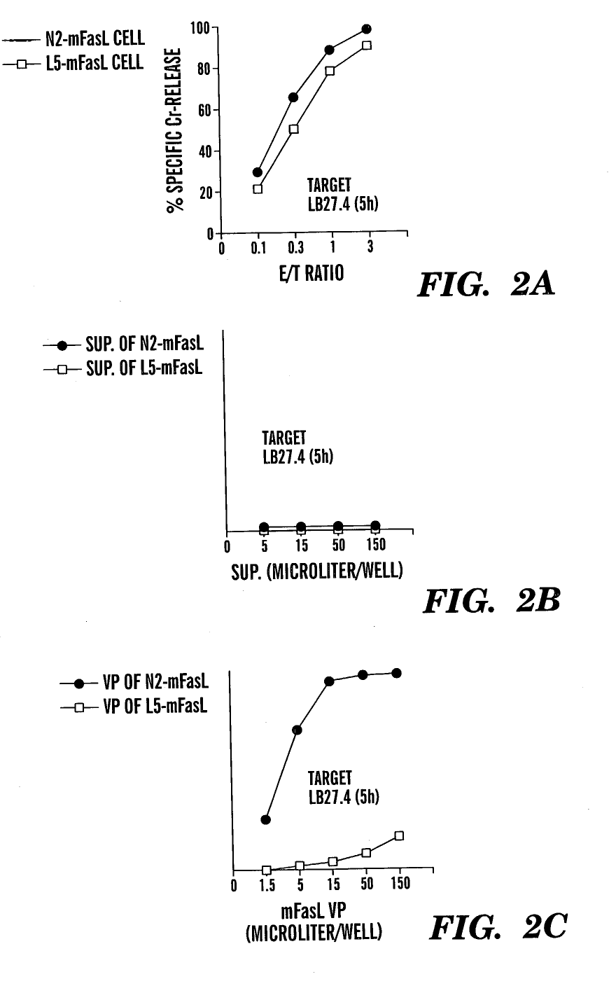

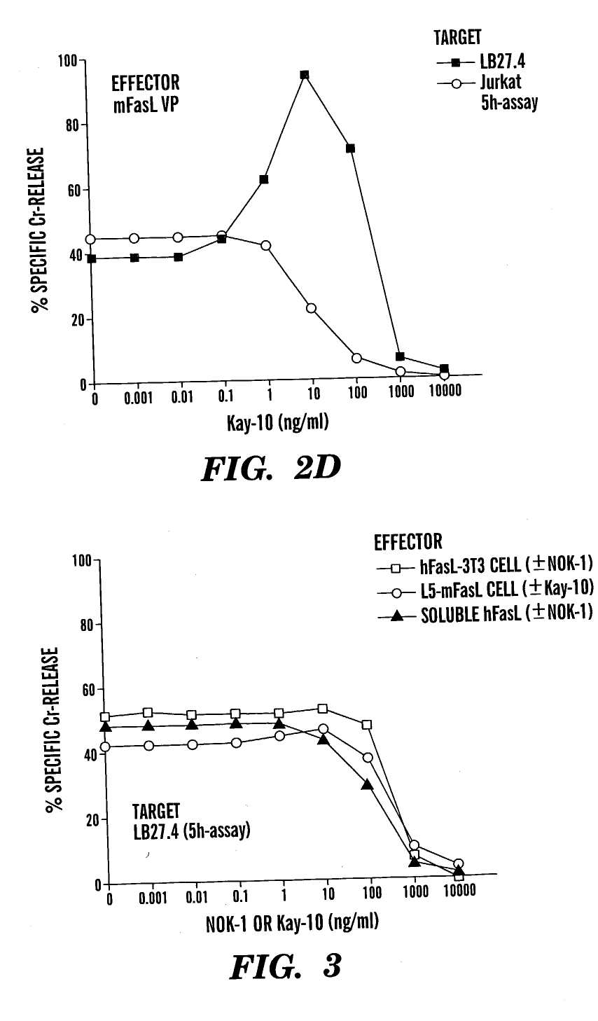

We have also prepared FasL VP from N2-mFasL and L5-mFasL cell lines, that expresses a form of mouse FasL in which the metalloproteinase sensitive site has been deleted (13). The cytotoxicity of FasL VP and supernatant (obtained following ultracentrifugation) was determined using LB27.4 target cells (FIG. 2). The cytotoxicity of cellassociated FasL was also determined. Both cell lines expressed strong cell-mediated cytotoxicity (FIG. 2a). As predicted, cytotoxic activity was not detected in the supernatant (FIG. 2b). Interestingly, FasL VP of N2-mFasL expressed strong cytotoxicity whereas little cytotoxicity was detected in the FasL VP prepared from L5-mFasL (FIG. 2c), suggesting variability in the production of bioactive vesicles among different cell lines (Jodo, S. et al, unpublished observation). Therefore, we tested Kay-10 mAb, which is specific for the mouse FasL (21), for the ability to modulate the cytotoxic activity of FasL VP prepared from N2-mFasL cells (FIG. 2d). We observed a similar pattern of enhancement and inhibition with LB27.4 target cells. Moreover, the dose ranges that respectively enhanced and inhibited the cytotoxicity were similar to that described in FIG. 1. Furthermore, inhibition but not enhancement, was observed when cytotoxicity was assessed with Jurkat target cells. Taken together, these studies consistently demonstrate enhancement of FasL VP-mediated cytotoxicity by a variety of FasL-specific antibodies with several cellular targets and several FasL VP preparations in multiple experiments.

Anti-FasL Ab-mediated enhancement of cytotoxicity is unique to FasL VP

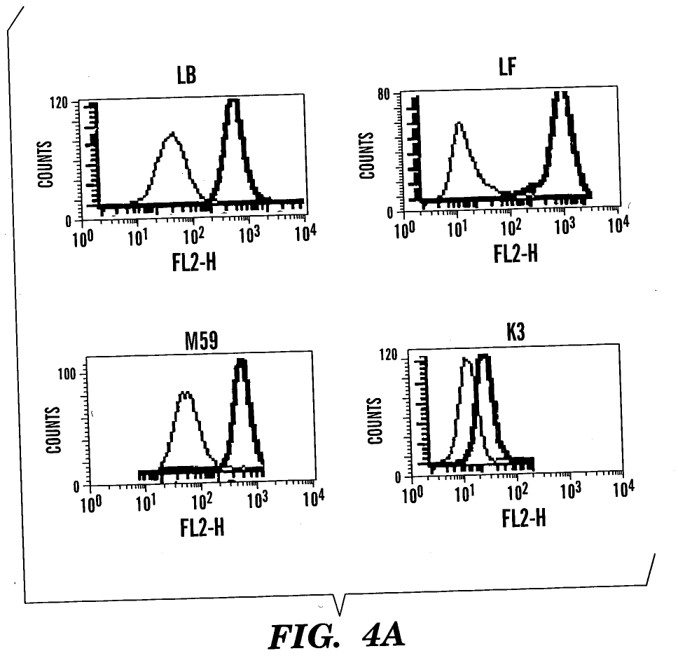

In an early study, we examined the ability of NOK-1 to modulate the cytotoxicity of cell-associated FasL and sFasL against LB27.4 target cells (10). We found that 1-100 ng/ml NOK-1 weakly enhanced the cytotoxicity of hFasL-PA317 cells. A similar result was obtained with N2-mFasL (data not shown). Because both cell lines efficiently produce FasL-expressing bioactive vesicles, the weak enhancement may not be the property of cell-associated FasL. We have examined a panel of FasL-expressing cell lines and found two (hFasL-3T3 and L5-mFasL) that do not produce a significant level of FasL VP during the 5-hour cytotoxicity assay. Therefore, the ability of NOK-1 and Kay-10 to modulate the cytotoxic activity of hFasL3T3 and L5-mFasL was determined (FIG. 3). The results were similar in both cases. Inhibition of cytotoxicity was observed with mAb concentration between 1-10 μg/ml. In contrast to the enhancement observed with FasL VP, the anti-FasL mAb, in a range between 1 -100 ng/ml, did not enhance the cell-mediated cytotoxicity against LB27.4 target cells. In addition, as previously described, inhibition (at the high dose range) but no enhancement (at the low dose range) of human sFasL cytotoxicity was observed with NOK-1 mAb (FIG. 3). These results indicate that only FasL VP have the capacity to work in concert with anti-FasL mAb and result in enhancement of cytotoxicity.

Anti-FasL Ab-mediated enhancement of FasL VP cytotoxicity depends on FcR expression on targets

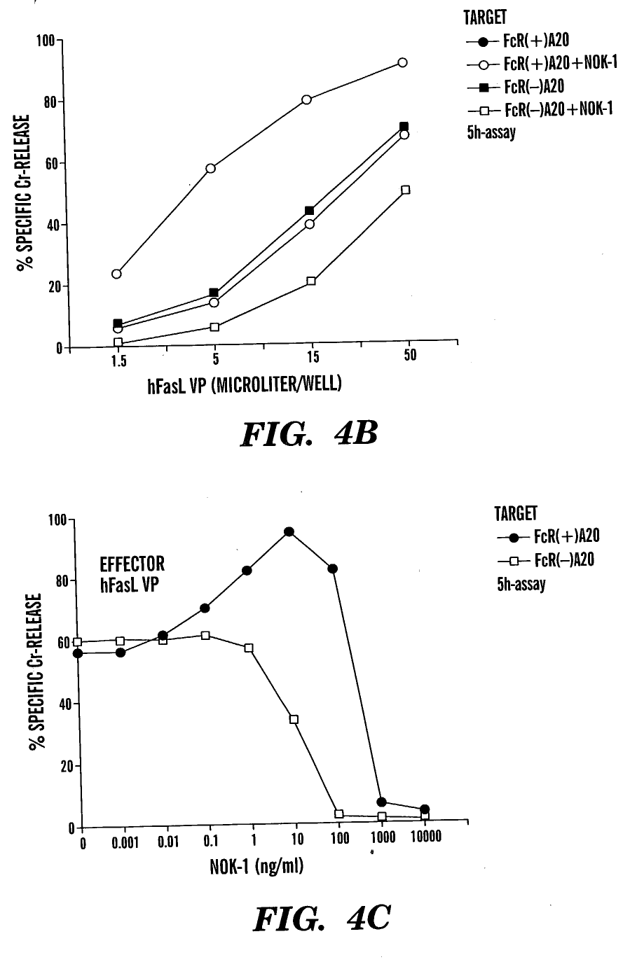

Since previous studies have shown that Jurkat cells do not express the FcR (23), we hypothesized that the enhancement of FasL VP cytotoxicity by anti-FasL mAb was mediated by Fc/FcR interactions that focus bioactive vesicles to the target cells. Whether the enhancement of cytotoxicity correlated with target FcR expression was determined by fluorescent staining using the FITC-conjugated 2.4G2 anti-FcR mAb. The results showed a specific and strong staining of LB27.4, LF+, and M59 cells (target populations that exhibit “enhanceable cytotoxicity”). In contrast, staining of K3 cells, the “non-enhanceable target population”, was extremely weak (data not shown). To further demonstrate that the enhancement of FasL VP cytotoxicity depends on FcR expression, we compared the effect of NOK-1 mAb on the cytotoxicity of FasL VP against FcR+ A20 and FcR− A20 target populations. As shown in FIG. 4a, FasL VP killed both targets, and the sensitivity of the two targets was comparable. In the presence of 10 ng/ml NOK-1 mAb, killing of FcR+ A20 was significantly enhanced. The enhancement was observed over a wide range of the FasL VP concentrations examined. In contrast, killing of the FcR− A20 target by FasL VP was inhibited (FIG. 4a). We also determined the effect of various doses of NOK-1 mAb on FasL VP cytotoxicity. While a dose-dependent inhibition was observed with the FcR− A20 target, the killing of FcR+ A20 was enhanced by low doses of NOK-1 mAb and inhibited by high doses of NOK-1 mAb (FIG. 4b). Enhancement but not inhibition of cytotoxicity is blocked by 2.4G2 anti-FcR mAb

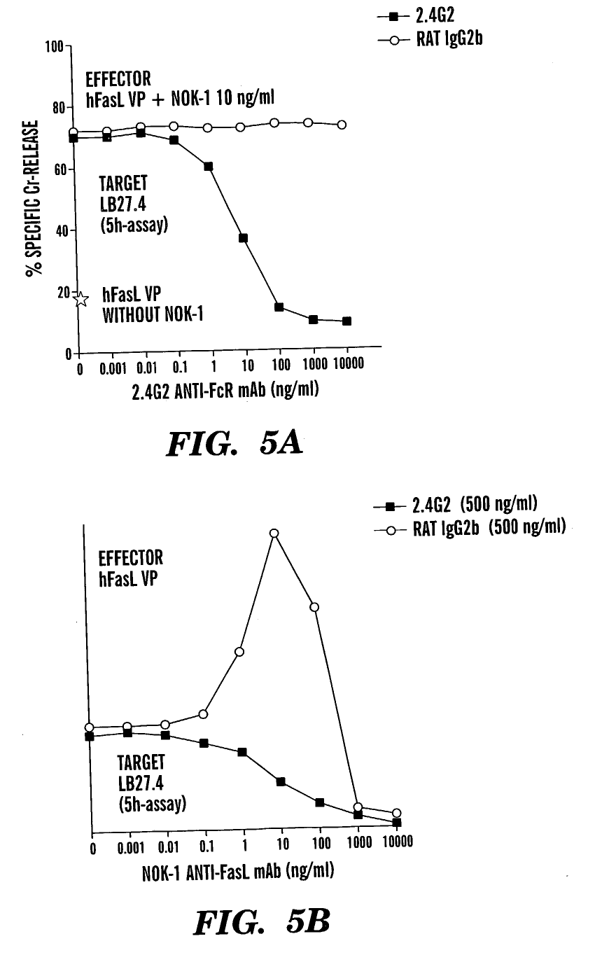

To directly establish the role of target FcR in the enhancement of FasL VP-mediated cytotoxicity, we used the 2.4G2 anti-FcR mAb to determine whether enhancement of cytotoxicity could be blocked (FIG. 5). As shown in FIG. 5a, 10 ng/ml of NOK-1 mAb increased the FasL VP-mediated cytotoxicity against 51 Cr-labeled LB27.4 cells from 19% to 72%. The presence of 2.4G2 mAb inhibited this enhancement of cytotoxicity in a dose-dependent manner. The enhancement was completely blocked by 100 ng/ml of 2.4G2 mAb, while a significant blocking was still observed with 2.4G2 mAb at a concentration of 10 ng/ml. No effect was observed with a normal rat IgG2b control mAb. Next, we determined the ability of various doses of NOK-1 to modulate the cytotoxicity of FasL VP in the presence of excess 2.4G2 mAb (500 ng/ml). The results show that the enhancing effect of NOK-1 mAb was completely blocked such that the entire peak of enhancement was eliminated. In contrast, the ability of NOK-1 mAb to inhibit cytotoxicity was not blocked (FIG. 5b). In addition to eliminating the enhancing effect of NOK-1 mAb, 2.4G2 appeared to facilitate the ability of NOK-1 mAb to inhibit cytotoxicity. This is shown by the ability of NOK-1 in the low dose range (1-100 ng/ml) to effectively inhibit cytotoxicity in the presence of 2.4G2 mAb. Influence of anti-FasL antibody subclasses and specificity on FasL VP-mediated cytotoxicity

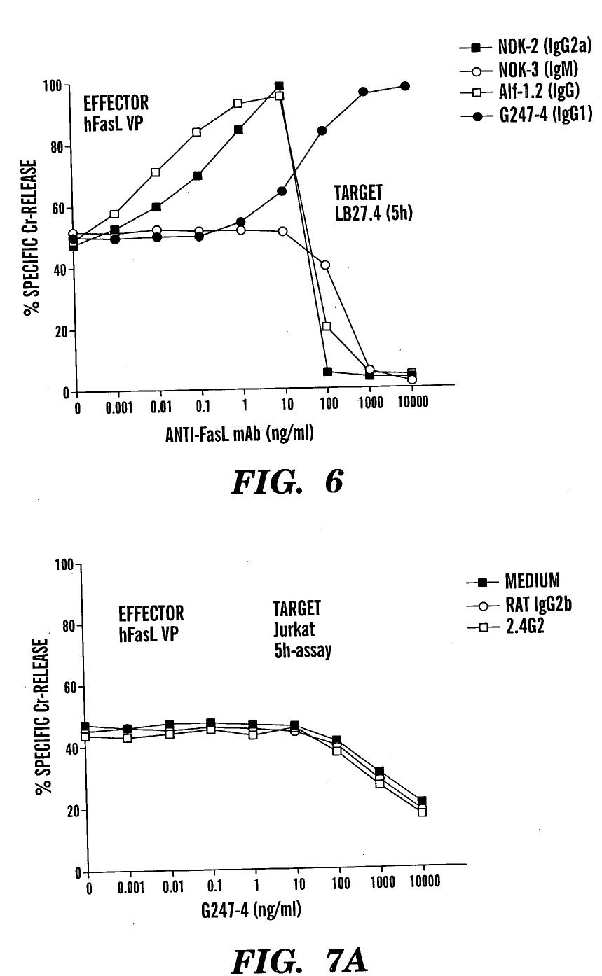

We tested a panel of anti-FasL mAb to determine the effect of anti-FasL mAb subclass and specificity on FasL VP cytotoxicity against the FcR+ LB27.4 target cells (FIG. 6). The same response pattern, i.e., enhancement with low concentrations and inhibition with high concentrations, was observed with NOK-2 and Alf-1.2 mAb, as had been seen with NOK-1 mAb (FIG. 1). An IgM anti-FasL mAb (NOK-3) did not enhance cytotoxicity at the low dose range but did inhibit cytotoxicity at the high dose range, consistent with the fact that the enhancement was dependent on the Fc of the IgG and not the IgM class of the FasL-specific mAb. Interestingly, the IgG1 mAb, G247-4, enhanced the FasL VP-mediated cytotoxicity even at the high dose range. Under identical condition, G247-4 inhibited the FasL VP-mediated cytotoxicity against Jurkat target cells (FIG. 7a). Next, we determined whether the G247-4 mediated enhancement of FasL VP cytotoxicity against LB27.4 target cells could be blocked by 2.4G2 mAb. The presence of 2.4G2 not only eliminated the enhancement but also allowed G247-4 to inhibit the cytotoxicity against LB27.4 (FIG. 7b). The conversion from enhancement to inhibition was not observed with rat IgG2b control. The presence of 2.4G2 did not influence the ability of G247-4 to block the killing of Jurkat cells (FIG. 7a). It was noted that the ability of G247-4 to inhibit cytotoxicity was weaker than the other mAb (compare with FIG. 6). G247-4 binds to a region near the “self-association” site (14). Such binding may not be as effective as binding to the FasL binding site for blocking cytotoxicity. It is also possible that G247-4 has a low affinity for FasL. The data nevertheless demonstrate that both the subclass and the fine specificity of the anti-FasL mAb are important parameters for modulating the cytotoxicity of FasL VP.

Effect of Fas-IgG1 Fusion Protein on FasL VP-mediated Cytotoxicity

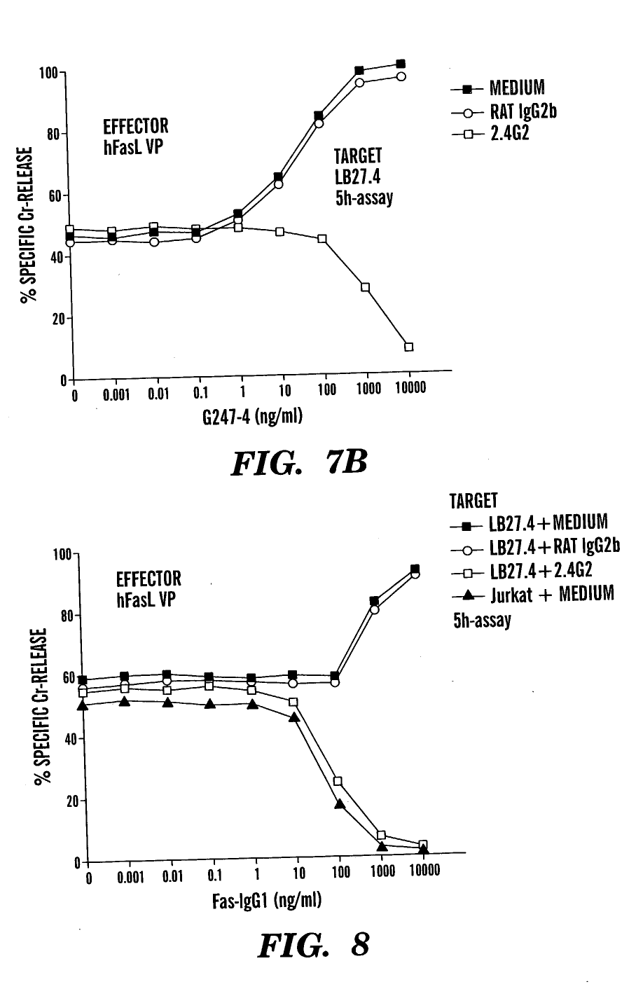

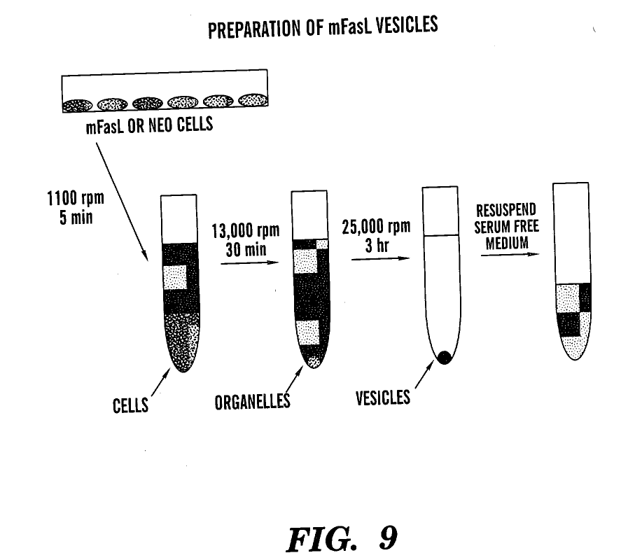

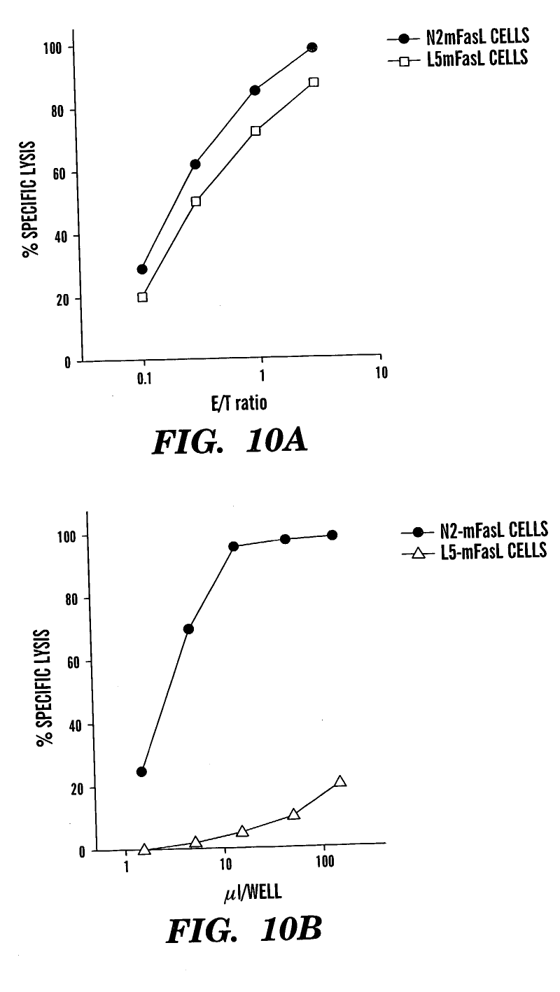

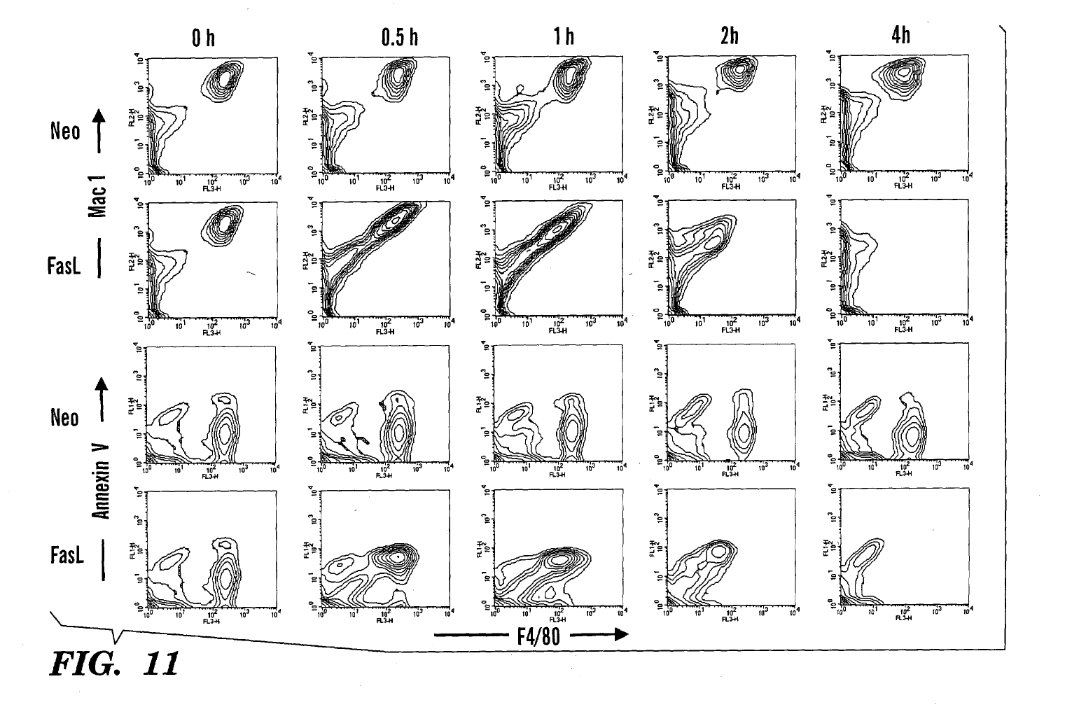

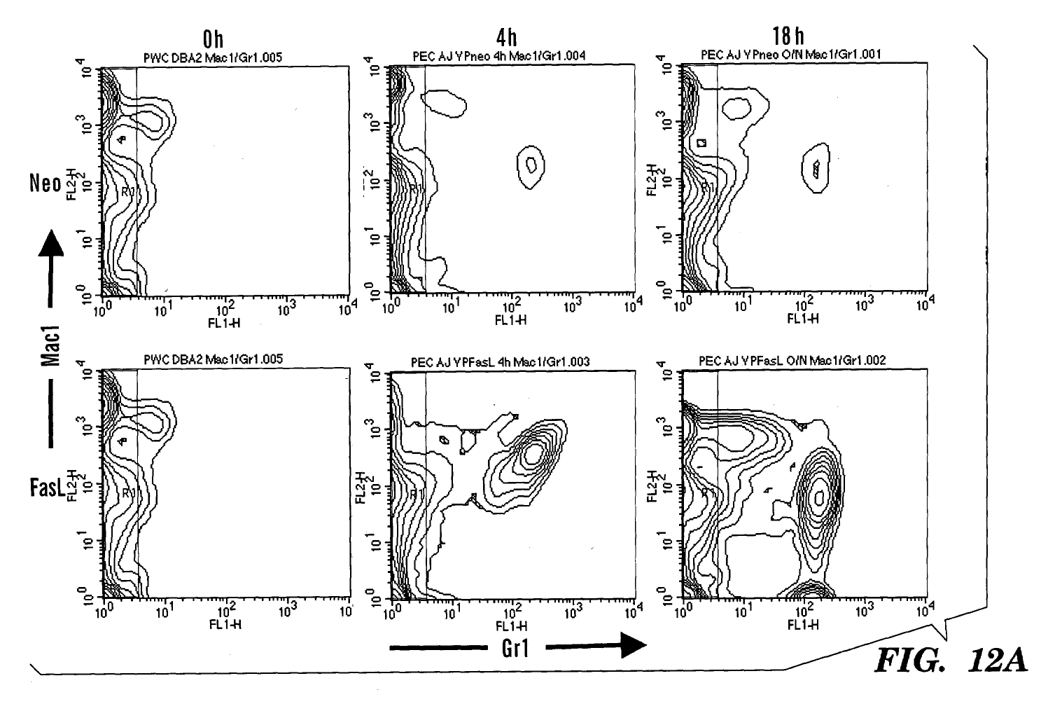

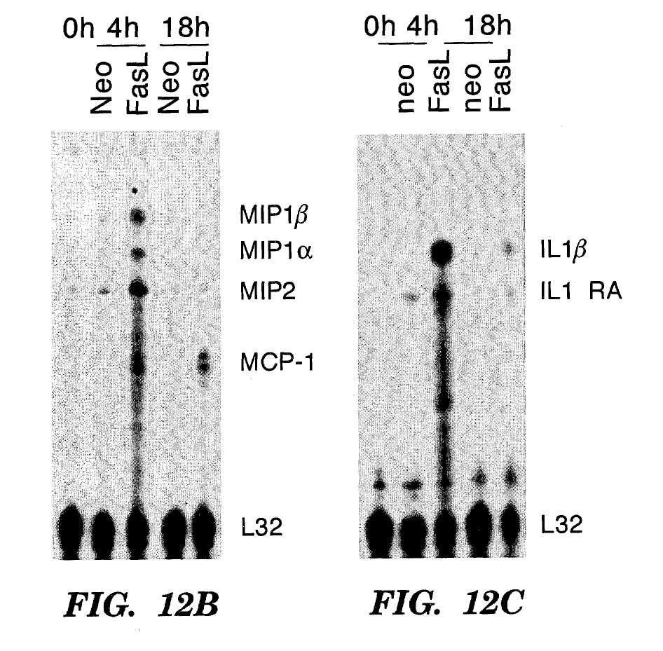

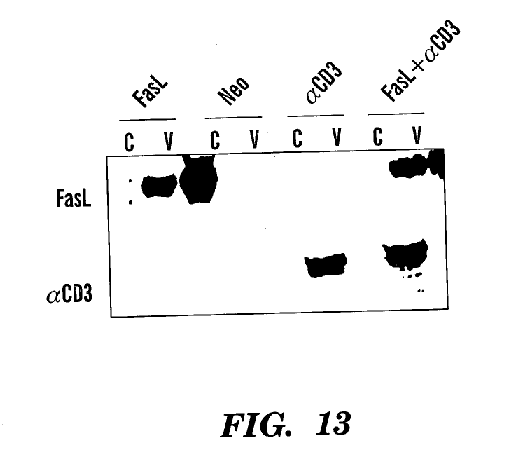

The Fas moiety on Fas-IgG1 fusion protein is a dimer rather than the trimer of natural Fas. Therefore, it interacts with the FasL binding site with a lower affinity than cell-associated Fas. Fas-IgG1 could be used to determine unambiguously whether enhancement of FasL VP-mediated cytotoxicity could be achieved with a molecule that binds to FcR (through IgG1 Fc) and FasL binding site (through Fas dimer). Therefore, we determined the effect of Fas-IgG1 on the cytotoxicity mediated by FasL VP against Jurkat and LB27.4 targets. The results shown in FIG. 8 indicate that Fas-IgG1 is a strong inhibitor of FasL VP when cytotoxicity was assessed on Jurkat target cells. In contrast, strong enhancement but no inhibition of cytotoxicity was observed with LB27.4 target cells. Like G247-4, enhancement of cytotoxicity was observed with high concentrations of Fas-IgG1. Moreover, 2.4G2, but not a control antibody, converted the Fas-IgG1-mediated enhancement of cytotoxicity to inhibition of cytotoxicity. Cytotoxicity was inhibited more than 50% with 100 ng/ml of Fas-IgG1 and complete inhibition of cytotoxicity was obtained with 1-10 μg/ml of Fas-IgG1. Taken together, the data demonstrate that FcR-dependent enhancement of FasL VP-mediated cytotoxicity significantly reduces the inhibitory ability of Fas-IgG1, even though the Fas-IgG1 interacts directly with FasL binding sites.

This study describes the novel effect of a panel of IgG anti-FasL mAb (NOK-1, NOK-2, Alf-1.2, and Kay-10) that can significantly enhance the cytotoxicity of FasL VP. We found that although most of these antibodies inhibited FasL VP cytotoxic activity at high concentrations, they enhanced cytotoxicity when used at low concentrations with target cells that were FcR+. The enhancement could be specifically blocked with the 2.4G2 anti-FcR mAb. In addition, the enhancing effect of these mAb was observed with FasL VP but not with sFasL or cell-associated FasL. The data establish that the enhancement is mediated by the interactions between the Fc of the anti-FasL mAb and the FcR on the target cell surface. Presumably, this interaction focuses the FasL-expressing bioactive vesicles onto the target cells, thereby increasing the cytotoxicity of FasL VP.

The fact that enhancement of cytotoxicity was observed only with FasL VP and at a relatively low concentration of anti-FasL mAb (1-100 ng/ml) indicates this phenomenon depends on the unique physical properties of the FasL VP and a critical concentration of anti-FasL that facilitates association between FasL VP and the target population but does not block all FasL binding sites. The small size of FasL VP as microvesicles (<0.45μ in diameter) in comparison to cells suggests that binding of a few anti-FasL mAb molecules would allow the focusing of FasL VP to LB27.4 through Fc/FcR binding. Increasing the concentration of the anti-FasL mAb blocks most or all FasL binding sites and results in inhibition of cytotoxicity.

There may be several possible reasons why mAb-mediated enhancement was not observed with cell-associated FasL. First, effector cells may use other interaction molecules such as integrins to facilitate their engagement with targets. Second, the interaction between vesicles and cells may rely more on Brownian's movement. Brownian's movement, coupled with the FcR-mediated focusing onto target cells, could provide faster and more effective Fas/FasL interaction in the latter case. Third, the amount of anti-FasL mAb needed to link an effector cell with a target may be so large that it is within the dose range that inhibits FasL cytotoxicity.

The results obtained with sFasL are consistent with the idea that multi-valency of the FasL is a critical factor. It has been suggested that recombinant sFasL, consisting of the entire extracellular domain, could exist in a multimeric form as ˜300 kDa aggregates, or about four trimeric units of FasL (14). We have shown that natural sFasL is heterogeneous and can be separated by filtration into two fractions, one with molecular masses between 100-500 kDa, and the other between 50-100 kDa (10). Thus, sFasL may have limited valency. If so, it is not surprising that binding of NOK-1 anti-FasL mAb to natural sFasL could not effectively focus a significant level of “non-inhibited” sFasL on LB27.4 to enhance cytotoxicity. In addition to NOK-1, inhibition but not enhancement of sFasL cytotoxicity was observed with NOK-2, NOK-3, and Alf-1.2 (data not shown). These results suggest that FasL VP particles are more multivalent than sFasL and display the appropriate physical form necessary to enhance cytotoxicity against FcR+ targets.

Once bound to FcR on target cells, there are two possible mechanisms whereby FasL VP might deliver its apoptotic signal. The focused FasL VP could either cross-link Fas on the very cell to which they attached or they could cross-link Fas on a neighboring bystander. In preliminary studies, enhancement by NOK-1 mAb was observed when cytotoxicity was conducted with 51Cr-labeled FcR+ A20 in the presence of either unlabeled FcR+ A20 or unlabeled FcR− A20 cells. Enhancement was not observed when cytotoxicity was conducted with 51Cr-labeled FcR− A20 in the presence of unlabeled FcR+ A20. These observations strongly suggest that NOK-1 mAb only enhances FasL VP-mediated cytotoxicity on target cells with functional FcR and the bound FasL VP does not enhance killing of neighboring bystanders. FcR may mediate enhancement simply by binding the Fc of NOK-1 mAb and thereby increasing the effective concentration of FasL VP at the cell surface. Alternatively, cross-linking FcR may transduce a signal that sensitizes the Fas-mediated death pathway. However, FcR mediates inhibitory signal in B cells but activation signal in T cells and macrophages (24), yet enhancement of cytotoxicity is observed in FcR+ target independent of this dichotomy. In addition, enhancement by NOK-1 mAb was not observed with sFasL and cell-associated FasL. Furthermore, a high dose of Genestein (0.2 mM) known to inhibit protein tyrosine phosphorylation did not affect FasL VP cytotoxicity against LB27.4 target cells and had no effect on the enhancement of FasL VP cytotoxicity by NOK-1 mAb (data not shown).

Distinct protocols were used to generate the panel of anti-FasL mAb used in this study. The fine specificity of some of these mAb has been analyzed (14). NOK-1 and G247-4 are thought to recognize the FasL binding site (the C-terminal region) and a segment near the “self-association” site (amino acid residues 103 to 136, non-binding site), respectively (14). The observation that high concentrations of G247-4 enhance the cytotoxicity of FasL VP suggests that the enhancement of FasL VP activity is dependent on the fine specificity or the affinity of the anti-FasL mAb. The latter interpretation was supported by similar results obtained with Fas-IgG1, which displays Fas as dimer and interacts with FasL binding site with a lower affinity than natural Fas. It should be noted, however, that both binding of G247-4 to the non-FasL binding site and binding of Fas-IgG1 to the FasL binding site resulted in inhibition of cytotoxicity if the FcR binding was blocked by 2.4G2 or if cytotoxicity was assayed on the FcR-Jurkat cells. These data suggest that reagents specific for either binding site or non-binding site epitopes of FasL possess the ability to enhance and inhibit cytotoxicity of FasL VP.

Although we could not demonstrate a significant enhancing effect of NOK-1 anti-FasL mAb on the cytotoxicity of cell-associated FasL, several observations suggest that FcR-mediated interactions reduce the ability of anti-FasL mAb to efficiently block cytotoxicity. First, the amount of the NOK-1 anti-FasL mAb needed to inhibit the killing of Jurkat cells was less than that needed to inhibit the killing of LB27.4 target (FIGS. 1 and 3). Second, the amount of NOK-1 anti-FasL mAb needed to inhibit the killing of LB27.4 was significantly reduced in the presence of 2.4G2 (compare FIG. 1 and FIG. 5). Third, 2.4G2 mAb converted enhancement to inhibition when cytotoxicity assays were conducted in the presence of G247-4, i.e., the inhibition was observed in the dose range that enhanced cytotoxicity in the absence of 2.4G2 (FIG. 7b). Finally, we observed that the ability of NOK-1 anti-FasL mAb to inhibit the killing of LB27.4 by hFasL-3T3 was enhanced in the presence of 2.4G2 mAb (data not shown).

The demonstration of Ab-mediated, FcR-dependent enhancement of cytotoxicity represents a newly identified cytotoxic mechanism of the immune system. In addition to understanding the properties of the FasL-expressing bioactive vesicles, sFasL and cell-associated FasL, the enhancement of FasL VP-mediated cytotoxicity offers a very sensitive assay for detecting anti-FasL mAb. We have used this assay to test a panel of IgG anti-FasL mAb. Three mAb were able to enhance cytotoxicity at 0.01-1 ng/ml. There may be a physiological significance of this observation. It has been shown that a significant percentage (˜30%) of sera from patients of systemic lupus erythematosus (SLE) contain autoantibodies against FasL (25). It has also been shown that FasL bioactive vesicles could be produced from activated T cells (8), tumor cells, (26) and a number of transfected cell lines over-expressing FasL (9, 10). In view of the powerful enhancing ability of anti-FasL mAb (below ng/ml level), it is highly possible that anti-FasL-mediated enhancement of cytotoxicity against FcR+ targets could take place in patients with SLE or other autoimmune diseases.

Finally, our demonstration that the IgG anti-FasL mAb can focus FasL VP onto target cells through Fc/FcR interactions is reminiscent of several earlier studies of viral replication in FcR+ cells (27-29). Peiris et al have reported Ab-dependent enhancement of virus replication in FcR+ cells and they have demonstrated that the enhancement was blocked by anti-FcR Ab (27). Homsy et al, have shown that anti-HIV Ab enhance HIV infection in human cells and the enhancement of HIV infection was mediated through FcR and not CD4 (28). It is also known that neutralized Dengue virus displays residual infectivity mediated through high-affinity IgG FcR (29). Our study extends these observations from viral replication to the expression of apoptotic function of the FasL VP. In this respect, the FcR-dependent, antibody-mediated interaction between retroviral particles and target cells may be exploited for the enhancement of gene transfer in FcR+ cells. Our study also raises concerns on the potential effects of IgG/FcR interactions in using IgG fusion proteins and mAb (e.g. anti-FasL, anti-TNF-α, etc.) as modulating agents in general. More studies are needed to determine the physiological significance of this phenomenon and how to exploit the Ab-mediated, FcR-dependent enhancing effect on bioactive vesicles and virus for practical use.

### References

1. Takahashi, T., M. Tanaka, C. I. Brannan, N. A. Jenkins, N. G. Copeland, T. Suda, and S. Nagata. 1994. Generalized lymphoproliferative disease in mice, caused by a point mutation in the Fas ligand. Cell 76:969.

2. Lynch, D. H., M. L. Watson, M. L. Alderson, P. R. Baum, R. E. Miller, T. Tough, M. Gibson, T. Davis-Smith, C. A. Smith, K. Hunter, D. Bhat, W. Din, R. G. Goodwin, and M. F. Seldin. 1994. The mouse Fas-ligand is mutated in the gld mice and is part of a TNF family gene cluster. Immunity 1:131.

3. Chinnaiyan, A. M., K. O'Rourke, M. Tewari, and V. M. Dixit. 1995. FADD, a novel death domain-containing protein, interacts with the death domain of Fas and initiates apoptosis. Cell 81:505.

4. Muzio, M., A., M. Chinnaiyan, F. C. Kischkel, K. O'Rourke, A. Shevchenko, J. Ni, C. Scaffidi, J. D. Bretz, M. Zhang, R. Gentz, M. Mann, P. H. Krammer, M. E. Peter, and V. M. Dixit. 1996. FLICE, a novel FADD-homologous ICE/CED-3-like protease, is recruited to the CD95 (Fas/APO-1) death-inducing signaling complex. Cell 85:817.

5. Kayagaki, N., A. Kawasaki, H. Ebata, S. Ohlnoto, S. Ikeda, S. Inoue, K. Yoshino, K. Okumura, and H. Yagita. 1995. Metalloproteinase-mediated release of human Fas ligand. J. Exp. Med. 182:1777.

6. Tanaka, M, T. Itai, M. Adachi, and S. Nagata. 1998. Down-regulation of Fas ligand by shedding. Nature Medicine. 4:31.

7. Schneider, P., N. Holler, J-L. Bodmer, M. Hahne, K. Frei, A. Fontana, and J. Tschopp. 1998. Conversion of membrane-bound Fas (CD95) ligand to its soluble form is associated with downregulation of its proapoptotic activity and loss of liver toxicity. J. Exp. Med. 187:1205.

8. Martinez-Lorenzo, M. J., A. Anel, S. Gamen, I. Monleon, P. Lasierra, L. Larrad, A. Pineiro, M. A. Alava, and J. Naval. 1999. Activated human T cells release bioactive Fas ligand and APO2 ligand in microvesicles. J. Immunol. 163:1274.

9. Strehlow, D., S. Jodo, and S-T. Ju. 2000. Retroviral membrane display of apoptotic effector molecules. Proc. Nat. Acad. Sci. USA. 97: 4209.

10. Jodo, S., D. Strehlow, and S-T. Ju. 2000. Bioactivities of FasL-expressing retroviral particles. J. Immunol. 164: 5062.

11. Miller, A. D., D. G. Miller, J. V. Garcia, and C. M. Lynch. 1993. Use of retroviral vectors for gene transfer and expression. Methods Enzymol. 217:581-599.

12. Widom, R. L., I. Culic, J. Lee, and J. H. Kom. 1997. Cloning and characterization of hcKrox, a transcriptional regulator of matrix gene expression. Gene 198:407-420.

13. Hohlbaum, A., M. S. Moe, and A. Marshak-Rothstein. 2000. Opposing effects of transmembrane and soluble Fas-ligand expression on inflammation and tumor cell survival. J. Exp. Med. 191: 1209.

14. Orlinick, J. R., K. B. Elkon, and M. V. Chao. 1997. Separate domains of the human Fas ligand dictate self-association and receptor binding. J. Biol. Chem. 272:32221.

15. Rouvier, E., M.-F. Luciani, and P. Golstein. 1993. J. Exp. Med. 177: 195.

16. Ju, S-T., D. J. Panka, H. Cui, R. Ettinger, M. El-Khatib, D. H. Sherr, B. Z. Stanger, and A. Marshak-Rothstein. 1995. Fas (CD95)/FasL interactions required for programmed cell death after T-cell activation. Nature 371:444.

17. Ju, S-T., and Dorf. M. E. 1985. Functional analysis of macrophage hybridomas. IV. Induction and inhibition of mixed lymphocyte responses. J. Immunol. 134:3722.

18. Jones, B., Tite J. P., and C. A. Jr. Janeway. 1986. Different phenotypic variants of the mouse B cell tumor A20/2J are selected by antigen- and mitogen-triggered cytotoxicity of L3T4-positive, I-A-restricted T cell clones. J. Immunol. 136:348.

19. Ju, S-T., N. H. Ruddle, P. Strack, M. E. Dorf, and R. H. DeKruyff. 1990. Expression of two distinct cytolytic mechanisms among murine CD4 subsets. J. Immunol. 144: 23.

20. Smith, D., S. Sieg, and D. Kaplan. 1998. Technical note: Aberrant detection of cell surface Fas ligand with anti-peptide antibodies. J. Immunol. 160:4159.

21. Kayagaki, N., N. Yamaguchi, F. Nagao, S. Matsuo, H. Maeda, K. Okumura, and H. Yagita. 1997. Polymorphism of murine Fas ligand that affects the biological activity. Proc. Natl. Acad. Sci. USA 94:3914.

22. Unkeless, J. C. 1979. Characterization of a monoclonal antibody directed against mouse macrophage and lymphocyte Fc receptors. J. Exp. Med. 150:580.

23. Wirthmueller U., T. Kurosaki, M. S. Murakami, and J. V. Ravetch. 1992. Signal transductin by FcγRIII (CD 16) is mediated through the y chain. J. Exp. Med. 175:1381.

24. Daeron M. 1997. Fc receptor biology. Annu. Rev. Immunol. 15: 203.

25. Suzuki, N., M. Ichino, S. Mihara, S. Kaneko,. and T. Sakane. 1998. Inhibition of Fas/Fas ligand-mediated apoptotic cell death of lymphocytes in vitro by circulating anti-Fas ligand autoantibodies in patients with systemic lupus erythematosus. Arthritis & Rheumatism. 41:344.

26. Albanese, J., S. Meterissian, M. Kontogiannea, C. Dubreuil, A. Hand, S. Sorba, and N. Dainiak. 1998. Biologically active Fas antigen and its cognate ligand are expressed on plasma membrane-derived extracellular vesicles. Blood 91:3862.

27. Peiris J. S., S. Gordon, J. C. Unkeless, and J. S. Porterfield. 1981. Monoclonal anti-Fc receptor IgG blocks antibody enhancement of viral replication in macrophages. Nature 289:189.

28. Homsy, J., M. Meyer, M. Tateno, S. Clarkson, and J. A. Levy. 1989. The Fc and not CD4 receptor mediates antibody enhancement of HIV infection in human cells. Science 244:1357.

29. Schlesinger, J. J., and S. E. Chapman. 1999. Influence of the human high-affinity IgG receptor FcγRI (CD64) on residual infectivity of neutralized Dengue virus. Virology. 260: 84-88.

The references cited in the specification are herein incorporated by reference in their entirety.

We claim:

1. A method of producing vesicles containing a functional membrane protein comprising the steps of:
(a) introducing a nucleic acid encoding the membrane protein into a cell capable of producing vesicles;
(b) selecting cells that express said nucleic acid and wherein the functional membrane protein is bound to a vesicle of the cell;
(c) growing the cells in culture medium under conditions that allow expression of the nucleic acid resulting in the functional membrane protein;
(d) collecting the culture medium;
(e) centrifuging the culture medium to remove cellular debris from the cell-free supernatant;
(f) centrifuging the cell-free supernatant to pellet the vesicles; and
(g) discarding the supernatant and resuspending the pellet containing the isolated vesicles containing the functional membrane protein in an isotonic solution.

2. The vesicle of claim 1, wherein the membrane protein is a type II transmembrane protein which is a member of the tumor necrosis factor (TNF) superfamily.

3. The vesicle of claim 2, wherein the type II transmembrane protein which is a member of the TNF superfamily is selected from the group consisting of Fas-ligand, TRAIL, RANKL, TWEAK, TNF-α, LTα:, LIGHT, LTα1β2, TL1A, CD40-ligand, CD30-ligand, CD27L, 4-1BBL, OX40L, GITRL, APRIL, BAFF, EDA1, and EDA2.

4. The vesicle of claim 3 wherein the protein is Fas-ligand.

5. The vesicle of claim 3 wherein the protein is tumor necrosis factor.

6. The vesicle of claim 3 wherein the protein is TRAIL.

7. A vesicle comprising at least two functional membrane proteins.

8. The vesicle of claim 7 wherein the membrane-bound proteins are Fas-ligand and an antibody.
